SEQUENCE LISTING (5 sequences)

SEQ ID NO: 1 - DNA, 1978 bp, Homo sapiens
  source 1..1978: mol_type = genomic DNA
ggggggtgcctgtggcactgcagaaagtgggcctgagcctcgaggatggcggtgctgcagggacccgtccaggctgctatatggcaagcactaaaccactatgcctaccgagatgcggtttcccccgcagaacgcctttatgcagaagtacactcagaagaagccttgttcttactggcgacctgttcttaccgctcaggaaaggcctataaaacatatagactcttgaaaggacacagttgtactaccccgcaatgcaaatgcctgcttgcgaaatgttgtgttgatctcagcaagcttgcagaaggggatcaaatcttatcgggtggagtgtttcataagcagaaaagccacgatgatattatcgttcctgagtttggtgattcagctcgctttccccttccatcgtcgggacatgtatattgcaagacagatcggcttaccaaaggatcagaatgtgaccaaaagagccttcgttgaaatcctttcctctggtgtccctttgaatcatgatgtgaaataggggggaaaagccagatcctgaccaaacattttgattcacctctctagagagctttagcaactgcctgtccaagtgttgcgccacaacacttggacctcgtcctagtttatgtcacagacagcccgagaccgttcttacggaaacaccccaggacaccgtcgaatgaaacggatggaatttagaatcttccaattcaaagtactccttgaatacagactccccagtgtcttctatcgattcagctgtcctttcacctgatactgtcccactgggaacaggaacttccatatgatctaaacaggttcaacataaaccaaaaactggtcgaagtttattaggaggaccagcagctgttagtccattaaccccaagtgttggcattttgccattagaaaccccaagtcccggagacggaccctattgacaaaactgcactggtatgagacaccgtctgtcactgacgtgccatccaccggagccccttcagaaaagcctgttgccagaatcggccaaaccggaacaccgtctgtcttctcgcagaggggaaagagccgaaaggtaactccagtccttgcaaaaacacaaagttctggtccgccgacaagggcaacacctcaggtattgagccccactatggcatctcccccaaatgcaccgcctcgaagaaattcacgactcttgactagtgacagctccacaaccgaggagaataggagaaaattaaaaatgaagtttccacctaaaatcccaaacagaagaaccaaaagtaaaactaataaaggagcaataactccaccgaacataagtgatagcctggaagtgacaaaattggactcttccgtcatttcagaagggaaaatagccgcaatcgcgcctcagattcaggcttttaatctacaaaaagcagcagcggaaggcttgctgagccttcttcgtgaaagggggaaaggttatttagctttgtgttcttaccacggcaaagaagctatcagcatcttgagccatctagcttctcaccactgcaataccggttgggtactgtgccaaatcggaagggcctattgtaaactttcagagtccacgcaagctgaaagacaattctcagaggttaggaggattgagaattacagagtcgaaggcatggagatctactctacaacactttggcatcctccgaaagaggttgctctttcagttctgtccaaagacttcacagacatggataaaaattcgccagccagaggcctggtgtgctgcagggaactgtttcggtctgcaacgggaacacgatattgcgattcaattcttccagagaactctcgaagtggatccaaatgatgcttatgcctatagcgcattagggcgtgagcttgtcttcactgaagaactggacaaagcattagcttgttttcgaaatgctatcagggtcaatcctagacattgtaaggcatggta

SEQ ID NO: 2 - DNA, 2993 bp, Homo sapiens
  source 1..2993: mol_type = genomic DNA
ctggcgtgctgcagggaaactgtttcggtctgcaacgggaacacgatattgcgattcaattcttccagagaactctcgaagtggatccaaatgatgcttatgcctatagcgcattagggcgtgagcttgtcttcactgaagaactggacaaagcattagcttgttttcgaaatgctatcagggtcaatcctagacattgtaaggcatggtaagtgctaatgaagcgtaaagacaaagccctatggatggtgccggtactcgctaatttttctggttagatagctctttattgtcacggatttggtgaaaaatacttagggatggtacctactgctgaataacttctaactaagatgtttccttacgaaacgtatgtcttgaacaaactctgaagtgaactcatgatcgtagaataccagatccttatactcaacagtttcagtcttctagcaaacttttgcagacgctgtagttgtctttggtttgtgtgtgtgtttctttagttgtgttccttgatttgttactttttcttcgagcaccgaagtggtgatggggacaagaagtgcttgggagactggaaaggaatagcatagttcacttattggataatagaaaaatacatggaaacaattcactagctgctgctttttgacagtgttccagtttacggagttactatgaagaacttcacgtaccctttaatttagcagtctctctgttttactcttttgtactcgtgtataagtaggcacataggaaattactacctaggtcatattgttatcaactgaataagataggaaaaagtgtggtcctacttctgcctcaacaccatcctcaccgttgacatttattgcgtttctctggactgacttcatagtttaaacgtcaagagaaggccgggctcagtggctcacgcctgtcatcccagcactttgggaggccgaggcgggcgggtcacgaggtcaagagatcgagaccatccgggccgacacggtgaaaccccgtctctattaaaagtataaagattagctgggcgtggtggcgggcacctgtagtcccagctactcgggaggctgaggcaggagaatcgcttgaacccagggaggtggcggttgcagtgagccgagatcgcaccatcgcactccagcctgggcgacagagcgagacgccgtctcaagagaaataaattaaaaaaaataaatacatacataagtaaatatcaagagaaagtatgattctgaagtcataaccctgtggtagttattttgtcagatacggtgatctttggggtgacttattacagcagtggagttctatcatttgatttgcttctaaatctgaagcattatattactgaaacactttttgatttgcgaatatgttgtttaatggatcatatctcattttgctgtagtagttacattgcccgaaagatggccaaaaagatagtgccagctactgctgaccaacgtaacaatcaacttgccaatactgccttctcttccgatagctacgttctccgtcctattttaagaactcagttcttcataagacttgtgtggttttcgattttttcccaagtctggttgatccttgtgttgttatttttttaaatgtgtatcgtctgttcagctattttgcaggagtcgcattcttaaaaaaatcttaaccctatcaaaaattgtgtttgtttaaaggaggattattcagatcggccagcttttactaggaagagtgtaaatgctgacgtatttaggtagctctaaatactgagcaactttattctaaccacaaaatagatagcctttcttttgtcttcactttcactatcattagcacagtgtttaataccgtttcttcatctataacacaattataatgatataggaagccactcaaataaggcagacatgttgcgttgcgcttaaaaaaaaaaaaaaaagaagtctctctgtggcacggaatgaggtgtggctcgaatctagaatctccagtgaaaaccaatgaaagagggtgaaaccccgtgtctaccaaaaaaaaaaaaaaaaaaaaaaaatgagccggccatggtggcgctgagacaggagaatcacttgaacccaggaggcagaggttccagtgagctgagatcacgccactgcactccagcctgggggacagagcaagactccatctcagaaacaaacaaacacacaaagccagtcaaggtgtttaattcgacggtgtcaggctcaggtctcttgacaggatacatccagcacccgggggaaacgtcgatgggtggggtggaatctattttgtggcctcaagggagggtttgagaggtagtcccgcaagcggtgatggcctaaggaagcccctccccccaagaagcgatattcatttctagcctgtagccacccaagagggagaaccgggctcgccacagaccccacaacccccaccccaccccacccccacccctcccacctcgtgaaatgggctctcgctccgtcaggctctagtcacaccgtgtggttttggaacctccagcgtgtgtgcgtgggttgcgtggtggggtggggccggctgtggacagaggaggggataaaggggcggtgtcccgcgggtgcccgggacgtggggcgtggggcgtgggtggggtggccagagccttgggaactcgtcgcctgtcgggacgtctcccctcctggtcccctctctgacctacgctccacatcttcgccgttcagtggggaccttgtgggtggaagtcaccatccctttggactttagccgacgaaggccgggctcccaagagtctccccggaggcggggccttgggcaggctcacaaggatgctgacggtgacggttggtgacggtgatgtacttcggaggcctcgggccaatgcagaggtatccatttgacctcggtgggacaggtcagctttgcggagtcccgtgcgtccttccagagactcatccagcgctagcaagcatggtcccgag

SEQ ID NO: 3 - DNA, 904 bp, Homo sapiens
  source 1..904: mol_type = genomic DNA
ctggcgtgctgcagggaaactgtttcggtctgcaacgggaacacgatattgcgattcaattcttccagagaactctcgaagtggatccaaatgatgcttatgcctatagcgcattagggcgtgagcttgtcttcactgaagaactggacaaagcattagcttgttttcgaaatgctatcagggtcaatcctagacattgtaaggcatggtaagtgctaatgaagcgtaaagacaaagccctatggatggtgccggtactcgctaatttttctggttagatagctctttattgtcacggatttggtgaaaaatacttagggatggtacctactgctgaataacttctaactaagatgtttccttacgaaacgtatgtcttgaacaaactctgaagtgaactcatgatcgtagaataccagatccttatactcaacagtttcagtcttctagcaaacttttgcagacgctgtagttgtctttggtttgtgtgtgtgtttctttagttgtgttccttgatttgttactttttcttcgagcaccgaagtggtgatggggacaagaagtgcttgggagactggaaaggaatagcatagttcacttattggataatagaaaaatacatggaaacaattcactagctgctgctttttgacagtgttccagtttacggagttactatgaagaacttcacgtaccctttaatttagcagtctctctgttttactcttttgtactcgtgtataagtaggcacataggaaattactacctaggtcatattgttatcaactgaataagataggaaaaagtgtggtcctacttctgcctcaacaccatcctcaccgttgacatttattgcgtttctctggactgacttcatagtttgctagcaagcatggtcccgag

SEQ ID NO: 4 - DNA, 211 bp, Homo sapiens
  source 1..211: mol_type = genomic DNA
ctggcgtgctgcagggaaactgtttcggtctgcaacgggaacacgatattgcgattcaattcttccagagaactctcgaagtggatccaaatgatgcttatgcctatagcgcattagggcgtgagcttgtcttcactgaagaactggacaaagcattagcttgttttcgaaatgctatcagggtcaatcctagacattgtaaggcatggta

SEQ ID NO: 5 - DNA, 293 bp, Homo sapiens
  source 1..293: mol_type = genomic DNA
ggccgggctcagtggctcacgcctgtcatcccagcactttgggaggccgaggcgggcgggtcacgaggtcaagagatcgagaccatccgggccgacacggtgaaaccccgtctctattaaaagtataaagattagctgggcgtggtggcgggcacctgtagtcccagctactcgggaggctgaggcaggagaatcgcttgaacccagggaggtggcggttgcagtgagccgagatcgcaccatcgcactccagcctgggcgacagagcgagacgccgtctcaagagaaataaa

(19) United States
(12) Patent Application Publication
(10) Pub. No.: US 2023/0340499 A1
(43) Pub. Date: Oct. 26, 2023

(54) CONSTRUCTS, COMPOSITIONS, CELLS AND METHODS FOR INCREASED RECOMBINANT PROTEIN EXPRESSION BY TARGETED INTEGRATION AND AMPLIFICATION

(71) Applicant: INPAMAC BIOTECH CANADA INC., Charlottetown (CA)

(72) Inventor: Laszlo Robert KATONA, Szeged (HU)

(21) Appl. No.: 18/149,913

(22) Filed: Jan. 4, 2023

Related U.S. Application Data
(63) Continuation of application No. PCT/IB2022/062155, filed on Dec. 13, 2022.
Application No. PCT/IB2022/062155 is a continuation-in-part of application No. PCT/IB2022/055963, filed on Jun. 28, 2022.
Application No. PCT/IB2022/055963 is a continuation-in-part of application No. PCT/IB2022/055964, filed on Jun. 28, 2022.
(60) Provisional application No. 63/254,997, filed on Oct. 12, 2021.
(60) Provisional application No. 63/215,876, filed on Jun. 28, 2021.

(51) Int. Cl. C12N 15/65; C12N 15/85

(52) U.S. Cl. CPC C12N 15/65; C12N 15/85; C12N 2800/24; C12N 2830/008

(57) ABSTRACT

Described are various embodiments of specific sequences for recombinant gene insertion, amplification and expression in the genomes of a variety of mammalian cells are disclosed. A DNA vector containing one or more such sequence enables high levels of recombinant gene expression following transfection, stable integration and amplification. The selection and cloning of the specific loci and the expression of recombinant genes is disclosed, as are the DNA vectors and the host cells.

### CROSS REFERENCE TO RELATED APPLICATIONS

The instant application claims priority to PCT Application No. PCT/IB2022/055963 filed Jun. 28, 2022 and entitled “COMPOSITIONS, CONSTRUCTS, CELLS AND METHODS FOR INCREASED RECOMBINANT PROTEIN EXPRESSION BY SITE SPECIFIC INTEGRATION”, and to PCT Application No. PCT/IB2022/055964 filed Jun. 28, 2022 and entitled “COMPOSITIONS, CONSTRUCTS, CELLS AND METHODS FOR CELL THERAPY”, the entire disclosures of each of which are hereby incorporated herein by reference.

The instant application is a Continuation-in-Part application of PCT Application No. PCT/IB2022/055963 filed Jun. 28, 2022 and entitled “COMPOSITIONS, CONSTRUCTS, CELLS AND METHODS FOR INCREASED RECOMBINANT PROTEIN EXPRESSION BY SITE SPECIFIC INTEGRATION”, which claims priority to claims priority to U.S. Provisional Patent Application Ser. No. 63/215,876 filed Jun. 28, 2021 and entitled “COMPOSITIONS, CONSTRUCTS, CELLS AND METHODS FOR INCREASED RECOMBINANT PROTEIN EXPRESSION BY SITE SPECIFIC INTEGRATION”, and to U.S. Provisional Patent Application Ser. No. 63/254,997 filed Oct. 12, 2021 and entitled “COMPOSITIONS, CONSTRUCTS, CELLS AND METHODS FOR CELL THERAPY”, the entire disclosures of each of which are hereby incorporated herein by reference. Additionally, the instant application is also a Continuation-in-Part Application of PCT Application No. PCT/IB2022/055964 filed Jun. 28, 2022 and entitled “COMPOSITIONS, CONSTRUCTS, CELLS AND METHODS FOR CELL THERAPY”, which claims priority to U.S. Provisional Patent Application Ser. No. 63/215,876 filed Jun. 28, 2021 and entitled “COMPOSITIONS, CONSTRUCTS, CELLS AND METHODS FOR INCREASED RECOMBINANT PROTEIN EXPRESSION BY SITE SPECIFIC INTEGRATION”, and U.S. Provisional Patent Application Ser. No. 63/254,997 filed Oct. 12, 2021 and entitled “COMPOSITIONS, CONSTRUCTS, CELLS AND METHODS FOR CELL THERAPY”, the entire disclosures of each of which are hereby incorporated herein by reference.

### SEQUENCE LISTING

The instant application contains a Sequence Listing which has been submitted electronically in XML format and is hereby incorporated by reference in its entirety. Said XML copy, created on Jan. 2, 2023, is named, “03250872.XML” and is 11 KB in size.

### FIELD OF THE DISCLOSURE

The present disclosure relates to gene expression, and, in particular, to compositions, constructs, cells and methods for increased recombinant protein expression by targeted integration and amplification.

### BACKGROUND

Approximately 20 to 30% of new drugs approved by the USFDA in recent years are biologics (in 2019 60 biologics were approved by CDER and CBER), up from 1 approval in 2000 (Batta et al., J Family Med Prim Care. 2020 January; 9(1): 105-114). There are currently more than 350 therapeutic biologics on the market and over 900 biologics in development. Development of expression systems for the efficient production of recombinant proteins is important for providing a source of a given protein for research or therapeutic use. The increased number of biologics in development has driven the need to develop simple and rapid high-output technologies for the development of recombinant protein expressing cell lines. The generation of commercial cell lines using conventional methods is a time-consuming, labor-intensive and repetitive process. The instant disclosure provides novel methods and materials that solve or ameliorate many of these significant problems.

Expression systems have been developed for both prokaryotic cells and for eukaryotic cells, which include yeast, Pichia pastoris, insect and mammalian cells. Expression in mammalian cells, for example Chinese hamster ovary (or “CHO”) cells, is often preferred for the manufacture of therapeutic proteins, since post-translational modifications in such expression systems are more likely to resemble those found in human cells expressing proteins than the type of post-translational modifications that occur in microbial (prokaryotic) expression systems. Additionally, baby hamster kidney (BHK21) and murine (prokaryotic) expression systems. Additionally, baby hamster kidney (BHK21) and murine myeloma cells (NS0 and Sp2/0) cells are used. Human cell lines like HEK293, HT1080, Per.C6, HKB-11, CAP, HuH-7 and other well-known cell lines are even more preferred. One skilled in the art can also develop means of using pluripotent, induced pluripotent, totipotent, adult stem cell lines, cultured adult cells and immortalized cells in certain embodiments of the disclosure.

Recombinant expression plasmids comprising a gene of interest that encodes all or a portion of a desired protein are routinely used to generate CHO cells expressing the desired recombinant protein. These recombinant plasmids randomly integrate into the genome of the host producing recombinant proteins but the frequency of cell lines carrying the stably integrated recombinant gene that are capable of expressing a desired recombinant protein at high levels is extremely low. A large number of transfected mammalian cell lines must be screened to identify clones which express the recombinant proteins at high levels. During the construction and selection of protein-producing cells lines, cell lines with a large range of expression, growth and stability profiles are obtained. These variations can arise due to the inherent plasticity of the mammalian genome. They can also originate from stochastic gene regulation networks or in variation in the amount of recombinant protein produced resulting from random genomic integration of a transgene principally due to the “position variegation effect” or merely from the plasmid copy number, especially in light of the large size of the mammalian genome and the fact that only a small percentage of the genomic DNA contains transcriptionally active sequences. Random integration also results in insertion mutagenesis which may cause unwanted changes that influences cell survival, cell proliferation, impairment in protein production, cell senescence, silencing etc.

As a consequence of these variations and the low (perhaps 1 in 10,000) frequency of genomic integration, resource-intensive and time-consuming efforts are required to screen many transfectants in the pool for these rare events, in order to isolate a commercially compatible production cell line (e. g., a combination of good growth, high productivity and stability of production, with desired product profile).

Expression augmenting sequences have been disclosed to increase expression of recombinant protein for eukaryotic expression systems (see for example WO 97/25420). An increase in the frequency of high-level recombinant gene expressing cell lines would provide a much greater pool of high protein expressing cell lines to choose from. This task can be accomplished by generating homologous recombinant plasmids targeted to transcriptionally active sites as disclosed herein and by devising a means to select for such cell lines.

There are a number of well-known and common amplification methods used to improve the yield of protein expression in mammalian cell systems. Amplification of the dihydrofolate reductase gene (Dhfr) by methotrexate (Mtx) exposure is commonly used for recombinant protein expression in Chinese hamster ovary (CHO) cells. However, this method is both time- and labor-intensive, and the cells that are generated are frequently unstable in culture. Further, the DHFR/MTX system has a long development time (taking up to 6 months), and the selection pressure is indirect. In addition, there is a need for a mutant CHO cell line which has the DHFR alleles knocked-out. It is not practical when various cell lines are needed to be used (HEK293, HT1080, Per.C6, various types of stem cells etc.)

Another common amplification system is the GS/MSX system. The glutamine synthetase (GS) expression system has been used for decades but has numerous issues. L-Methionine sulfoximine (MSX) inhibits the activity of glutamine synthetase, an enzyme essential for the production of glutamine. MSX is used as a media supplement to aid selection and amplification processes in recombinant mammalian cell lines that use GS as a selective marker. However, this system also has a long development time, provides indirect selection pressure and the amplified gene to be expressed is even more unstable than the cell-lines developed by the DHFR/MTX system. In addition, there is a need for a mutant cell line which has the GS alleles knocked-out.

The failings of existing amplification systems are discussed in Joseph J. Priolal, Nathan Calzadillal, Martina Baumann2, Nicole Borth3, Christopher G. Tate4 and Michael J. Betenbaughl High-throughput screening and selection of mammalian cells for enhanced protein production Biotechnol. J. 2016, 11. p1-13. DOI 10.1002/biot.201500579.

One of the most common methods of recombinant protein production involves transfection with one of a variety of viruses including lentivirus, retrovirus, adenovirus and adeno-associated virus systems. See for example, Tandon, et al., Bio Protoc. 2018 Nov. 5; 8(21):. doi:10.21769/BioProtoc.3073. This approach has many issues:
1) Safety issues: A very high titer of infectious viruses carrying the gene of interest is required in a packaging cell line thus necessitating a high-level biosafety lab. Technicians must be highly trained to make and use these viruses, because the viruses can easily infect such technicians. The viruses may cause cancer or other problems because of virus integration triggered mutagenesis. Also, they have to wear special safety clothes, gloves, goggles etc. 17% of the human genome consists of long interspersed repeat element (“LINE”) sequences. These are hypothesized to be the remnants of previous virus infections. These viruses infected us and then some of them randomly integrated into the human genome. These virus sequences are a permanent part of the human genome and may even constitutively produce virus proteins such as reverse transcriptase. Reverse-transcriptase transcribes an RNA sequence and makes the DNA sequence from it in a reversal of the usual protein production process. This DNA sequence might then integrate into the human genome and cause mutations. Even if defected lentivirus vectors are used, such vectors might recombine with these pre-existing LINE sequences and result in an active virus. The use of virus vectors even for making cell lines poses potentially dangerous risks.
2) Purification issues. Proteins purified from such viral systems require time consuming additional purification steps in order to ensure that the protein does not contain virus or viral toxin contaminants.
3) Cell line stability. During the process of creating cell lines with a virus, the lentivirus integrates into the target genome randomly at hundreds or even thousands of sites. This random integration causes mutations in the cell line which can be advantageous for protein production in the short term but in the long term these mutations are harmful for the cell line. These methods are most useful for short term protein production. Over a longer term these randomly integrated viruses cease to produce proteins due to silencing. There is an evolutionary mechanism to silence integrated viruses. That is thought to be a natural mechanism our cells use to keep the LINE sequences at bay.
4) There is an ongoing technical problem with high-titer virus production since several plasmids must be co-transfected into the virus producing cell line. This co-transfection method is highly inefficient due to the fact that the various plasmids enter the cells in various concentrations, thus the plasmid which is transfected in the lowest amount is limiting virus production efficiency. Also, the virus producing cell lines must be genetically engineered to produce factors that are necessary for high-titer virus productions.
5) Virus vectors have limited carrying capacity.

To achieve a long-term supply of a recombinant protein, a random integration system is not optimal. Embodiments of the present disclosure provide more stable protein production in a safer, faster, long-lasting and easier system.

The technical problem underlying the present disclosure is to overcome the above-identified disadvantages, in particular to provide, preferably in a safe, simple and efficient manner, high producing cell lines with a high stability and positive growth and productivity characteristics, in particular cell lines which provide a consistent productivity over a long cultivation and production period.

In particular, the present disclosure solves or ameliorates much of these technical problems by the provision of a targeted integration (TI) host cell comprising an endogenous genomic amplification drop site(s) (GADS, which may also be referred to as genomic super expression drop site(s) or genomic overexpression drop site(s)), wherein an exogenous nucleotide sequence is integrated at said GADS. In some embodiments, the exogenous nucleotide sequence comprises at least one gene coding sequence of interest. In some embodiments, the nucleotide sequence comprises at least one selection marker gene, one promoter sequence and one enhancer sequence.

This background information is provided to reveal information believed by the applicant to be of possible relevance. No admission is necessarily intended, nor should be construed, that any of the preceding information constitutes prior art or forms part of the general common knowledge in the relevant art.

### SUMMARY

The following presents a simplified summary of the general inventive concept(s) described herein to provide a basic understanding of some aspects of the disclosure. This summary is not an extensive overview of the disclosure. It is not intended to restrict key or critical elements of embodiments of the disclosure or to delineate their scope beyond that which is explicitly or implicitly described by the following description and claims.

In accordance with one broad aspect of the disclosure, a nucleic acid construct is provided. The nucleic acid construct comprises a DNA fragment capable of targeted insertion into a region of open chromatin in mammalian cells on at least one chromosome at more than one site.

In one embodiment, the nucleic acid construct is amplified more than 5 times following selective pressure.

In one embodiment, the DNA fragment comprises the sequence of SEQ ID NO:1, SEQ ID NO:2, SEQ ID NO:3, SEQ ID NO:4, SEQ ID NO:5 or a functional fragment thereof.

In one embodiment, the DNA fragment is insertable at more than 5 sites on two or more chromosomes.

In accordance with another broad aspect of the disclosure, an expression vector comprising the nucleic acid construct as described above is provided.

In one embodiment, the expression vector further comprises a selectable marker. In one particular embodiment, the selectable marker provides resistance to puromycin.

In one embodiment, the expression vector further comprises a nucleic acid sequence encoding all or a portion of a protein of interest or the CDS for several proteins of interest.

In one embodiment, the protein of interest is a drug selected from the group consisting of Cetuximab, Omalizumab, Adalimumab, Abciximab, Abciximab, Infliximab, Trastuzumab, Rituximab, Basiliximab, Muromonab, Ibritumomab, Tositumomab, Alemtuzumab, Efalizumab, Palivizumab, Daclizumab, Bevacizumab, Arcitumomab, Eculizumab, Panitumumab, Canakinumab, Ipilimumab, Tocilizumab, Pertuzumab, Denosumab, Belimumab, Raxibacumab, Obinutuzumab, Natalizumab, Secukinumab, Gemtuzumab ozogamicin, Satumomab Pendetide, Alirocumab, Atezolizumab, Blinatumomab, Daratumumab, lotuzumab, Evolocumab, Idarucizumab, Vedolizumab, Ustekinumab, Siltuximab, Ramucirumab, Pembrolizumab, Ofatumumab, Obiltoxaximab, Nivolumab, Necitumumab, Mepolizumab, Ixekizumab, Brodalumab, Canakinumab, Dinutuximab, Ibritumomab tiuxetan, proteins of tumor biology, proteins of food industry, proteins of animal health, proteins of ageing, proteins of genetic disorders, vaccines, viral-like particles (VLPs), single proteins, virus inhibitor proteins, or the like.

In accordance with another broad aspect of the disclosure, a mammalian host cell transformed with the expression vector as described above is provided.

In one embodiment, the expression vector further comprises a selectable marker.

In one embodiment, the mammalian host cell is a HEK293 cell, a HT1080 cell, a Per.C6 cell, a HKB-11 cell, a CAP cell, a HuH7 cell, a pluripotent cell, an induced pluripotent cell, a totipotent cell, an adult stem cell, a primary cell, cultured adult cells or immortalized cells.

In accordance with another broad aspect of the disclosure, there is provided a Chinese hamster ovary (CHO) cell transformed with the expression vector described above.

In accordance with another broad aspect of the disclosure, there is provided a mouse cell transformed with the expression vector described above.

In accordance with another broad aspect of the disclosure, there is provided a human cell transformed with the expression vector described above.

In accordance with another broad aspect of the disclosure, there is provided a method for obtaining a recombinant protein, which comprises culturing a transformed host cell under conditions promoting expression of the recombinant protein and recovering the recombinant protein. In particular, the transformed host cell may be any one of the CHO cell, the mouse cell or the human cell, as described above.

In accordance with various other broad aspect of the disclosure, there is provided various plasmids constructed in accordance with various plasmid maps, typically including at least the nucleic acid construct described above.

In some embodiments, the plasmid further comprises a transgene and a selectable marker that provides resistance to puromycin.

In accordance with a further broad aspect of the disclosure, there is provided a DNA vector capable of integration into a mammalian genome. The DNA vector comprises a DNA fragment capable of targeted insertion into a region of open chromatin in mammalian cells, wherein the DNA fragment is the sequence of SEQ ID NO:1, SEQ ID NO:2, SEQ ID NO:3, SEQ ID NO:4, SEQ ID NO:5, or a functional fragment thereof; and at least one transgene encoding at least one protein of interest to be expressed by a mammalian cell after the DNA vector integrates into the mammalian genome of the mammalian cell.

In some embodiments, the at least one protein of interest is a drug selected from the group consisting of Cetuximab, Omalizumab, Adalimumab, Abciximab, Abciximab, Infliximab, Trastuzumab, Rituximab, Basiliximab, Muromonab, Ibritumomab, Tositumomab, Alemtuzumab, Efalizumab, Palivizumab, Daclizumab, Bevacizumab, Arcitumomab, Eculizumab, Panitumumab, Canakinumab, Ipilimumab, Tocilizumab, Pertuzumab, Denosumab, Belimumab, Raxibacumab, Obinutuzumab, Natalizumab, Secukinumab, Gemtuzumab ozogamicin, Satumomab Pendetide, Alirocumab, Atezolizumab, Blinatumomab, Daratumumab, lotuzumab, Evolocumab, Idarucizumab, Vedolizumab, Ustekinumab, Siltuximab, Ramucirumab, Pembrolizumab, Ofatumumab, Obiltoxaximab, Nivolumab, Necitumumab, Mepolizumab, Ixekizumab, Brodalumab, Canakinumab, Dinutuximab, Ibritumomab tiuxetan, proteins of tumor biology, proteins of food industry, proteins of animal health, proteins of ageing, proteins of genetic disorders, vaccines, viral-like particles (VLPs), single proteins, virus inhibitor proteins or the like.

Other aspects, features and/or advantages will become more apparent upon reading of the following non-restrictive description of specific embodiments thereof, given by way of example only with reference to the accompanying drawings.

### BRIEF DESCRIPTION OF THE FIGURES

Several embodiments of the present disclosure will be provided, by way of examples only, with reference to the appended drawings, wherein:

FIG. 1 shows a plasmid map of a recombinant insertion and expression vector designed to create cell lines that express the protein(s) of interest.

FIG. 2 shows a plasmid map of another recombinant insertion and expression vector designed to create cell lines that express trastuzumab (Herceptin, i.e. an insertion vector for trastuzumab heavy and light chain production).

FIG. 3 shows a fluorescent in situ hybridization (FISH) image of a trastuzumab producing CHO-K1 cell line, which cell line was produced in less than one month, wherein bright dots show the presence of trastuzumab expressing gene copies and wherein the stars show that amplification is clearly visible on a chromosome in two bands.

FIG. 4 shows an image of a Western blotting experiment of trastuzumab producing CHO-K1 cell lines, wherein the signal at 55 kDa is the trastuzumab heavy chain. Star depicts the protein production of the cell line for which the FISH experiment was shown on FIG. 3.

FIG. 5 shows an image of a Western blot of HEK293 cells showing the comparison of trastuzumab heavy chain production (55 kDa) and control actin production (42 kDa). In addition to the controls, trastuzumab production is shown in lanes labelled HETP10/5, HETP10/6, HETP10/8, and HETP10/13, wherein each lane corresponds to particular clones.

FIG. 6 shows an image of a Western blot showing purified trastuzumab production of both heavy (55 kDa) and light (25 kDa) chains from clone HETP10/13 of FIG. 5, wherein purification was done on protein G (SpinTrap, Merck) spin column purification (F1=first fraction of protein elution, F2=second fraction of protein elution).

FIG. 7 shows a Western blotting experiment showing the results of subcloning of clone HETP10/13, wherein the signal at 55 kDa is the trastuzumab heavy chain and the signal at 42 kDa is actin (internal control for quantitation of protein expression with Licor Odyssey gel documentation system).

FIG. 8 shows a FISH analysis image of HETP10/13 cell line's human chromosomes, wherein stars show targeted sites and amplification of trastuzumab genes.

FIG. 9 shows a plasmid map of a plasmid carrying GADS2 (SEQ ID NO:2) for transfecting, insertion and amplification.

FIG. 10 shows a FISH analysis image of FISH-stained MEF cells transfected with a GADS2 carrying plasmid according to the disclosure and as shown in FIG. 9, which in this embodiment carries the Influenza A virus Hemagglutinin MYMC_X-181 California strain sequence as a useful transgene, wherein targeting exclusively happened into a large acrocentric chromosome into the upper part of the long chromosomal arm in all 34 clones.

FIG. 11 shows a FISH analysis image of a CHO cell line's chromosomes where hamster chromosomes were stained with DAPI (blue signal), and the Influenza A virus Hemagglutinin MYMC_X-181 California strain sequence as a useful gene was labeled with a green fluorescent dye. The plasmid from FIG. 9 was used to make this cell line. It is clearly visible that in this cell line a new mammalian artificial chromosome was formed. The new MAC is visible beside a large hamster chromosome that carries hundreds of copies of the transgenes at the end of that chromosome. The newly formed MAC is almost exclusively composed of the delivered transgenes as the almost uniform green staining clearly demonstrates this.

FIG. 12 shows a plasmid map of a plasmid construct according to the disclosure carrying the GADS 1 sequence (CDC27 pseudogene) and the Influenza A virus Hemagglutinin MYMC_X-181 California strain sequence as a useful transgene.

FIG. 13 shows FISH experiment carried out with the plasmid on FIG. 12. Plasmid was labeled with a green fluorescent dye. Green staining shows the presence of the transgenes on a mouse chromosome in bands (several hundred copies). Mouse chromosomes were counterstained with DAPI (blue fluorescent dye).

FIG. 14 shows a plasmid map of a pIKRBBP7 plasmid for insertion and amplification of the RBBP7 protein.

FIG. 15 shows Western Blottting experiments of CIRBBP cell-lines generated using the pIKRBBP7 plasmid of FIG. 13, specifically showing expression of 6xHis and AVI tagged RBBP7 protein using an anti-AVI-tag antibody as a primary antibody and anti-mouse-HRP antibody as a secondary antibody in the Western blotting experiments. Western blotting experiments were developed by ECL and chemiluminescent signal was photographed.

Elements in the several figures are illustrated for simplicity and clarity and have not necessarily been drawn to scale. For example, the dimensions of some of the elements in the figures may be emphasized relative to other elements for facilitating understanding of the various presently disclosed embodiments. Also, common, but well-understood elements that are useful or necessary in commercially feasible embodiments are often not depicted in order to facilitate a less obstructed view of these various embodiments of the present disclosure.

### DETAILED DESCRIPTION

Various implementations and aspects of the specification will be described with reference to details discussed below. The following description and drawings are illustrative of the specification and are not to be construed as limiting the specification. Numerous specific details are described to provide a thorough understanding of various implementations of the present specification. However, in certain instances, well-known or conventional details are not described in order to provide a concise discussion of implementations of the present specification.

Various methods and processes will be described below to provide examples of implementations of the disclosure disclosed herein. No implementation described below limits any claimed implementation and any claimed implementations may cover processes or methods that differ from those described below. The claimed implementations are not limited to methods or processes having all of the features of any one method or process described below or to features common to multiple or all of the methods or processes described below. It is possible that a method or process described below is not an implementation of any claimed subject matter.

Furthermore, numerous specific details are set forth in order to provide a thorough understanding of the implementations described herein. However, it will be understood by those skilled in the relevant arts that the implementations described herein may be practiced without these specific details. In other instances, well-known methods, procedures and components have not been described in detail so as not to obscure the implementations described herein.

It is understood that for the purpose of this specification, language of “at least one of X, Y, and Z” and “one or more of X, Y and Z” may be construed as X only, Y only, Z only, or any combination of two or more items X, Y, and Z (e.g., XYZ, XY, YZ, ZZ, and the like). Similar logic may be applied for two or more items in any occurrence of “at least one . . . ” and “one or more . . . ” language.

Unless defined otherwise, all technical and scientific terms used herein have the same meaning as commonly understood by one of ordinary skill in the art to which this disclosure belongs.

As used herein, the term “transgene” is given its broadest possible meaning to include any gene that one wants to express regardless of the species or source of that gene. For example, a wild-type human gene or a mutant mouse gene could both be a transgene when used in the constructs and methods herein regardless of the species of the host cell, for that matter.

Throughout the specification and claims, the following terms take the meanings explicitly associated herein, unless the context clearly dictates otherwise. The phrase “in one of the embodiments” or “in at least one of the various embodiments” as used herein does not necessarily refer to the same embodiment, though it may. Furthermore, the phrase “in another embodiment” or “in some embodiments” as used herein does not necessarily refer to a different embodiment, although it may. Thus, as described below, various embodiments may be readily combined, without departing from the scope or spirit of the innovations disclosed herein. The same logic may apply to examples.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of singular forms “a,” “an,” and “the” include plural references unless the context clearly dictates otherwise. The meaning of “in” includes “in” and “on.”

The term “comprising” as used herein will be understood to mean that the list following is non-exhaustive and may or may not include any other additional suitable items, for example one or more further feature(s), component(s) and/or element(s) as appropriate.

Novel insertion sequences, referred to herein as GADS (genomic amplification drop site(s)), that facilitate increased expression of recombinant proteins in mammalian host cells, are disclosed. A preferred embodiment of the disclosure is a GADS that was obtained from human cell genomic DNA. Vectors comprising such human derived GADS can be used for insertion into not only human but also mouse and hamster genomes as a result of the highly conserved nature of the GADS. Alternatively, one skilled in the art can easily obtain mouse or hamster GADS sequences and other mammalian cell lines useful for protein expression could be envisioned by one skilled in the art. Another preferred embodiment of the disclosure is a GADS that was obtained from hamster cell genomic DNA. Another preferred embodiment of the disclosure is a GADS that was obtained from mouse cell genomic DNA.

The present disclosure discloses a GADS sequence from a genomic locus in the human genome that is capable of high recombinant gene amplification and expression. In a most preferred embodiment of the disclosure, the GADS is selected from the group consisting of (a) DNAs comprising nucleotides of SEQ ID NO:1; (b) fragments of SEQ ID NO:1 that are useful as insertion and expression sites; (c) nucleotide sequences complementary to (a) and/or (b); (d) nucleotide sequences that are at least about 80%, more preferably about 90%, and more preferably about 95% identical in nucleotide sequence to (a), (b) and/or (c) and that are useful for insertion and expression of exogenous proteins; and (e) combinations of the foregoing nucleic acid sequences that are useful for insertion and expression of exogenous proteins.

Expression vectors comprising the novel GADS sequences are able to transform CHO, HEK293, or other mammalian cells to increase expression of recombinant proteins through genomic insertion into open chromatin and amplification. Thus, another embodiment of the disclosure is an expression vector comprising a GADS sequence. In a preferred embodiment, the expression vector further comprises a eukaryotic promoter/enhancer driving the expression of all or a portion of a protein of interest. Two or more different nucleic acids expressing exogenous proteins of interest can be present in an expression vector used to transfect a cell (e.g., CHO or HEK293 cells), wherein each nucleic acid sequence encodes a different polypeptide that assemble (when expressed) to form a desired protein. In an additional preferred embodiment, the expression vector comprises a plasmid that encodes a gene of interest and also encodes an amplifiable dominant selectable marker. A preferred marker is puromycin; other amplifiable markers known in the art are also suitable for use in certain embodiments of the expression vectors of the instant disclosure.

Mammalian host cells can be transformed with an expression vector of the present disclosure to produce high levels of recombinant protein. Accordingly, another embodiment of the disclosure provides a mammalian host cell transformed with an expression vector of the present disclosure. Also within the scope of the present disclosure are mammalian host cells transformed with two expression vectors, wherein each of the two expression vectors encodes at least one polypeptide subunit that when co-expressed assembles into a desired protein with biological activity. In a most preferred embodiment, the host cells are CHO or HEK cells.

The disclosure also provides a method for obtaining a recombinant protein, comprising transforming a host cell with an expression vector of the present disclosure, culturing the transformed host cell under conditions promoting amplification of the inserted exogenous vector and expression of the protein, and recovering the protein. In a preferred application of the disclosure, transformed host cells are selected with multiple selection steps with increasing concentrations of a selection antibiotic such as puromycin. Embodiments of this method are useful for creating an amplification and expression system which is tunable to the specific properties of different transgenes that one desires to produce.

In certain embodiments of the disclosure, the selectable antibiotic (preferably puromycin) concentration is increased in a series of steps to achieve the desired optimal amplification and expression level in each cell type (preferably human, mouse or hamster). The methods, constructs and systems disclosed herein also provide a large number and variety of individual clones with various levels of amplification providing different expression levels. The particular clones having optimal amplification levels can be selected to provide a stable cell line with a desired protein production level. One skilled in the art will understand that different proteins require different levels of production and the methods disclosed herein (as well as the constructs and uses) provide an adjustable combination of integration, amplification and protein production. One skilled in the art can use the methods and constructs disclosed herein to adjust the system for the best results for each individual desired protein product.

The instant disclosure therefore comprises methods, compositions and constructs useful for production of large amounts of recombinant proteins in cell lines that are stable for long periods of time. Embodiments of the disclosure include sequences and constructs for achieving non-random insertion of exogenous DNA sequences into the genome of mammalian cell lines followed by the amplification of the inserted DNA into multiple sites across numerous chromosomes. In one surprising aspect of the instant disclosure, the non-random insertion sites of the disclosure are substantially composed of euchromatin that are open and not silenced. Thus, the constructs of the instant disclosure are uniquely useful for the stable, long-term, large scale production of therapeutic proteins.

Embodiments of the instant disclosure include methods for achieving rapid amplification of the inserted DNA across the genomes of the transformed cells.

Certain embodiments of the recombinant expression vectors of the instant disclosure include novel sequences for achieving homologous recombination at specific regions in the genomes of mammalian cells.

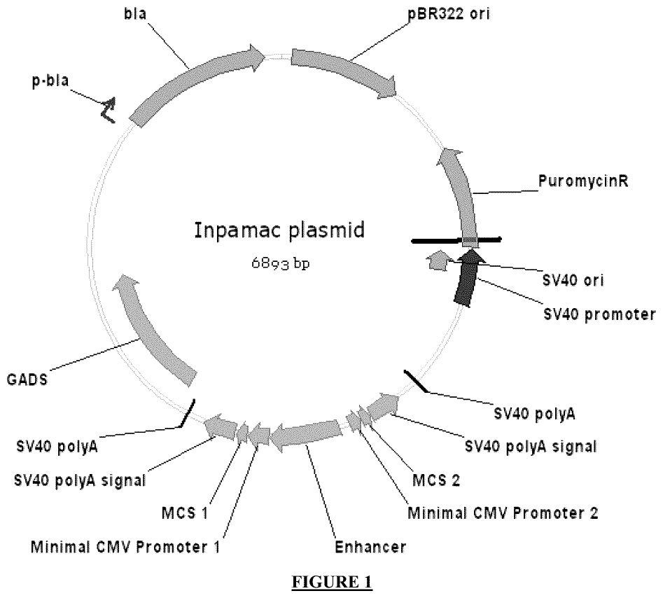

Recombinant expression vectors include synthetic or cDNA-derived DNA fragments encoding a protein, operably linked to suitable transcriptional or translational regulatory elements derived from mammalian, viral, fungi, insect or bacterial genes. Such regulatory elements may include a transcriptional promoter, a sequence encoding suitable mRNA ribosomal binding sites, and sequences which control the termination of transcription and translation. Mammalian expression vectors may also comprise non-transcribed elements such as an origin of replication, a suitable promoter and enhancer linked to the gene to be expressed, other 5′ or 3′ flanking non-transcribed sequences, 5′ or 3′ non-translated sequences such as ribosome binding sites, a polyadenylation site, and transcriptional termination sequences, secretion signals, various tag sequences. An origin of replication that confers the ability to replicate in a host, and a selectable gene to facilitate recognition of transformants, may also be incorporated. A preferred expression vector is shown in FIG. 1.

DNA regions are operatively linked when they are functionally related to each other. For example, a promoter is operatively linked to a coding sequence if it controls the transcription of the sequence; or a ribosome binding site is operatively linked to a coding sequence if it is positioned so as to permit translation. Transcriptional and translational control sequences in expression vectors used in transforming cells are known in the art.

Transformed host cells are cells which have been transformed or transfected with expression vectors constructed using recombinant DNA techniques and which contain sequences encoding all or a portion of recombinant proteins. Expressed proteins may be secreted into the cell culture supernatant, depending on the DNA selected, but may also be deposited inside the cell and/or in the cell membrane. Various mammalian cell culture systems can be employed to express recombinant protein according to embodiments of the present disclosure, all well known in the art, for example COS lines of monkey kidney cells, CHO cells, HeLa cells, HEK293 cells, Per.C6 cells, HKB-11 cells, CAP cells, HuH7 cells, LMTK-cells, NS0cells, Sp2/0 cells, pluripotent cells, induced pluripotent cells, totipotent cells, adult stem cells, primary cells, cultured adult cells and BHK cell lines.

Several transformation protocols are known in the art, and are reviewed, for example, in Kaufman et. al., (1988) Meth. Enzymology 185:537. The transformation protocol chosen will depend on the host cell type and the nature of the gene of interest and can be chosen based upon routine experimentation. The basic requirements of any such protocol are first to introduce DNA encoding a protein of interest into a suitable host cell, and then to identify and isolate host cells which have incorporated the DNA in stable, expressible manner. Examples of methods useful for introducing DNA encoding a protein of interest can be found in Wigler et.al., (1980) Proc. Natl. Acad. Sci. USA 77:3567; Schaffner (1980) Proc. Natl. Acad. Sci. USA 77:2163; Potter et.al, (1988) Proc. Natl. Acad. Sci. USA 81:7161; and Shigekawa (1988) BioTechniques 6:742.

A method of amplifying the gene of interest is also desirable for expression of the recombinant protein, and typically involves the use of a selection marker. The novel characteristics of the instant homologous recombination vectors are ideal for amplification using a selection marker. Resistance to cytotoxic drugs is the characteristic most frequently used as a selection marker and can be the result of either a dominant trait (i.e., can be used independent of host cell type) or a recessive trait (i.e., useful in particular host cell types that are deficient in whatever activity is being selected for). Many amplifiable markers are suitable for use in the present disclosure (for example, as described in Maniatis, Molecular Biology: A Laboratory Manual, Cold Spring Harbor Laboratory, NY (1989)). Useful selectable markers for gene amplification in drug-resistant mammalian cells include DHFR-MTX (methotrexate) resistance (Alt et.al., (1978) J. Biol. Chem. 253:1357; Wigler et. al., (1980) Proc. Natl. Acad. Sci. USA 77:3567), and other markers known in the art (as reviewed, for example, in Kaufman et.al., (1988) Meth. Enzymology 185:537). The most widespread method for amplifying a target gene in cell culture is the use of methotrexate (Mtx) treatment to amplify dihydrofolate reductase (Dhfr), however, surprisingly, embodiments of the present disclosure provide a significantly better amplification method using increasing concentrations of puromycin.

A preferred selection and amplification marker is the gene that encodes puromycin resistance. In certain embodiments of the present disclosure high levels of puromycin are used to apply selective pressure on the cells and the exogenous gene spreads along with the puromycin resistance gene throughout the GADS. In certain embodiments of the disclosure more than 5 copies of the exogenous gene are spread across different sites and chromosomes. In certain preferred embodiments, more than 10 copies of the exogenous gene are spread across different sites and chromosomes and in certain other preferred embodiments more than 25 copies of the exogenous gene are spread across different sites and chromosomes. In the most preferred embodiments, more than 50 copies of the exogenous gene are spread across different sites and chromosomes, leading to very high expression of the exogenous gene.

Previous methods of applying puromycin selection have been time consuming (see for example, Prieto et al., Prieto et al. BMC Proceedings 2011, 5(Suppl 8):P7 at www.biomedcentral.com/1753-6561/5/S8/P7) and methods of the present disclosure found surprising and unexpected results using a novel protocol of multi-step increases in puromycin concentration. Preferred methods of the disclosure involve step wise increases in puromycin from 5 ug/ml up to 350 ug/ml with each increase taking place after less than 7 days. In more preferred embodiments puromycin concentration was increased after about 3 days.

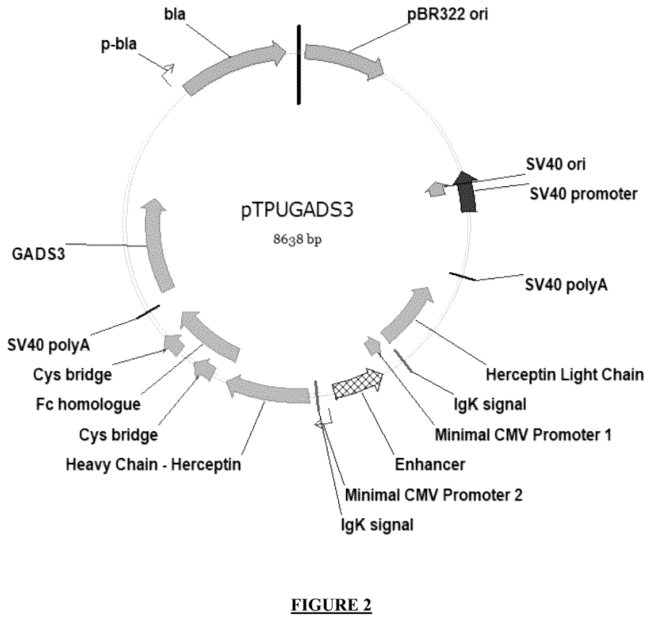

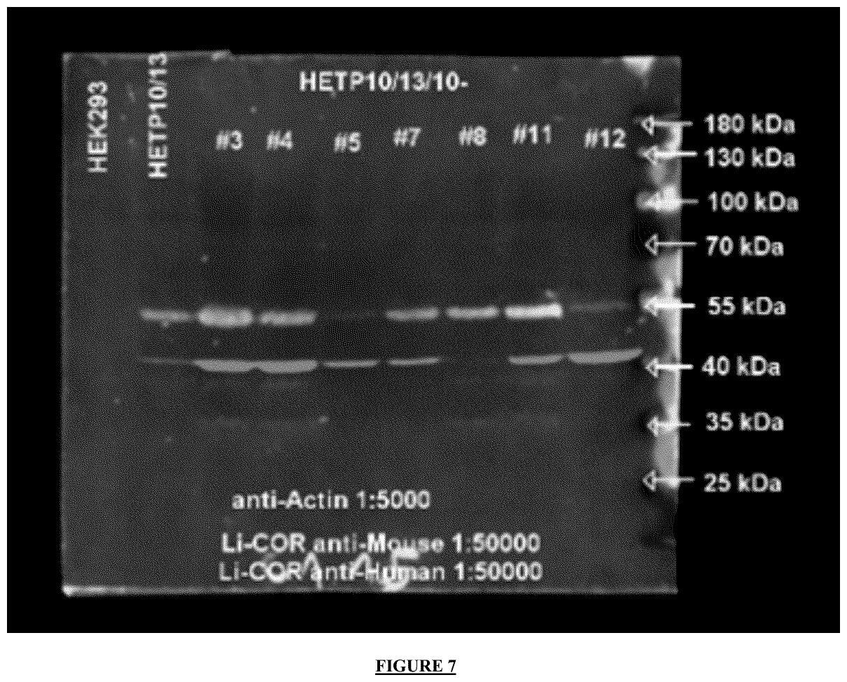

In certain embodiments of the present disclosure cell lines are created that express one recombinant protein or peptide of interest. In more preferred embodiments of the disclosure the cell lines of the disclosure produce two or more different proteins or peptides. In still further preferred embodiments, four different proteins or peptides may be produced in a single cell line. An example of the expression of two different peptides is provided herein as shown in FIG. 6 which depicts the production of both the heavy and light chains of the trastuzumab antibody following with transformation of HEK293 cells by the vector depicted in FIG. 2.

Thus, preferred embodiments of the expression vectors may encode one recombinant protein or peptide of interest. In more preferred embodiments of the expression vectors two or more different proteins or peptides are encoded. In still further preferred embodiments, four different proteins or peptides may be encoded in a single vector. In other embodiments, multiple vectors may be used to express yet more exogenous proteins or peptides.

In certain embodiments of the disclosure the recombinant proteins produced may be antibodies. In certain preferred embodiments, both light and heavy chains of antibodies may be produced by the same cell line.

Embodiments of the disclosure can be used to produce almost any protein, even complex biologics containing multiple polypeptide chains. In certain embodiments of the disclosure the cell lines stably produce large amounts of a drug selected from adalimumab, atezolizumab, nivolumab, pembrolizumab, etanercept, trastuzumab, bevacizumab, rituximab, aflibercept, infliximab, ustekinumab, ranibizumab, cetuximab, dornase alfa, peginterferon alfa-2a, darbepoetin alfa, reteplase, epoetin alfa, pegfilgrastim, thyrotropin alfa, antihemophilic factor, anistreplase, tenecteplase, coagulation factor viia, omalizumab, imiglucerase, abciximab, abciximab, interferon beta-1a, follitropin beta, basiliximab, muromonab, ibritumomab, tositumomab, alemtuzumab, laronidase, efalizumab, choriogonadotropin alfa, coagulation factor ix, agalsidase beta, palivizumab, daclizumab, arcitumomab, eculizumab, panitumumab, idursulfase, alglucosidase alfa, galsulfase, abatacept, canakinumab, ipilimumab, tocilizumab, pertuzumab, rilonacept, denosumab, belatacept, belatacept, velaglucerase alfa, brentuximab vedotin, taliglucerase alfa, belimumab, raxibacumab, epoetin zeta, obinutuzumab, follitropin alpha, romiplostim, natalizumab, secukinumab, drotrecogin alfa, alefacept, urokinase, gemtuzumab ozogamicin, satumomab pendetide, alirocumab, ancestim, antithrombin alfa, antithrombin iii human, asfotase alfa, blinatumomab, c1 esterase inhibitor (human), coagulation factor xiii a-subunit (recombinant), conestat alfa, daratumumab, lotuzumab, evolocumab, hyaluronidase (human recombinant), idarucizumab, vedolizumab, turoctocog alfa, simoctocog alfa, siltuximab, sebelipase alfa, ramucirumab, peginterferon beta-1a, ofatumumab, obiltoxaximab, necitumumab, mepolizumab, ixekizumab, brodalumab, c1 esterase inhibitor (recombinant, canakinumab, dinutuximab, efmoroctocog alfa, ibritumomab tiuxetan, lenograstim, pegloticase, protein s human, somatropin recombinant, susoctocog alfa, or thrombomodulin alfa.

Cetuximab, as disclosed in patent CA1340417, is an epidermal growth factor receptor which binds to FAB region. Cetuximab consists of the variable antigen-binding regions of the 225 murine EGFr monoclonal antibody that is specific for N-terminal part of human EGFr with human IgG1 heavy chain and kappa light chain constant regions. The sequence for this protein is disclosed at https://go.drugbank.com under accession number: DB00002.

Dornase alfa, as disclosed in patents CA2184581, CA2137237, is a biosynthetic form of human enzyme DNase I, produced in genetically modified CHO cells using recombinant DNA technology. The 260 amino acid synthetic sequence of dornase alfa is identical to the endogenous human enzyme. Dornase alfa cleaves extracellular DNA to 5′-phosphodinucleotide and 5′-phospho-oligonucleotide end products (without affecting intracellular DNA). In individuals with cystic fibrosis, extracellular DNA, an extremely viscous anion, is released by degenerating leukocytes which accumulate during inflammatory responses to infections. Enzymatic breakdown of this extracellular DNA causes reduction in sputum viscosity and viscoelasticity. The sequence for this protein is disclosed at https://go.drugbank.com under accession number: DB00003.

Etanercept, as disclosed in patents CA2476934, CA2123593, U.S. Pat. No. 7,276,477, US36755, is a dimeric fusion protein (934 amino acids) consisting of extracellular ligand-binding portion of the human 75 kilodalton (p′75) tumor necrosis factor receptor (TNFR) linked to Fc portion of human IgG1 produced by recombinant DNA technology in a Chinese hamster ovary (CHO) mammalian cell expression system. The Fc component of etanercept contains the CH2 domain, the CH3 domain and hinge region, but not the CH1 domain of IgG1. The sequence for this protein is disclosed at https://go.drugbank.com under accession number: DB00005.

Peginterferon alfa-2a, as disclosed in patents CA2203480, CA2172664, Human interferon 2a, is a covalent conjugate of recombinant interferon alfa-2a with a single branched bis-mono-methoxy polyethylene glycol (PEG) chain. The PEG moiety is linked at a single site to the interferon alfa moiety via a stable amide bond to lysine. Peginterferon alfa-2a has an approximate molecular weight of 60, 000 daltons. Interferon alfa-2a is produced using recombinant DNA technology in which a cloned human leukocyte interferon gene is inserted and expressed in Escherichia coli. The resultant protein is 165 amino acids. The PEG strand protects the molecule in vivo from proteolytic breakdown, substantially increases its in vivo half-life, and reduces immunogenicity by wrapping around and physically hindering access to the protein portion of the molecule. The sequence for this protein is disclosed at https://go.drugbank.com under accession number: DB00008.

Darbepoetin alfa, as disclosed in patents CA2165694, CA2147124, is Human erythropoietin with 2 amino acid substitutions to enhance glycosylation (5 N-linked chains), 165 residues (MW 37 kD) produced in CHO cells by recombinant DNA technology. The sequence for this protein is disclosed at https://go.drugbank.com under accession number: DB00012

Reteplase, as disclosed in patent CA2107476, Human tissue plasminogen activator which is glycosylated and purified (355 residues) from CHO cells. Retavase is considered a third-generation thrombolytic agent, genetically engineered to retain and delete certain portions of human tPA. Retavase is a deletion mutein of human tPA formed by deleting various amino acids present in endogenous human tPA. Retavase contains 355 of the 527 amino acids of native human tPA (amino acids 1-3 and 176-527), and retains the activity-related kringle-2 and serine protease domains of human tPA. Three domains are deleted from retavase—kringle-1, finger, and epidermal growth factor (EGF). The sequence for this protein is disclosed at https://go.drugbank.com under accession number: DB00015.

Epoetin alfa, as disclosed in patent CA1339047, is recombinant human erythropoietin which is produced by CHO cells. The sequence for this protein is disclosed at https://go.drugbank.com under accession number: DB00016.

Pegfilgrastim, as disclosed in patents CA1341537, CA1339071, is PEGylated(at N terminus) form of human G-CSF (Granulocyte colony stimulating factor), 175 residues, produced from E. coli via bacterial fermentation. The sequence for this protein is disclosed at https://go.drugbank.com under accession number: DB00019.

Thyrotropin Alfa, as disclosed in U.S. Pat. No. 5,840,566, is a recombinant form of thyroid stimulating hormone used in performing certain tests in patients who have or have had thyroid cancer. It is also used along with a radioactive agent to destroy remaining thyroid tissue in certain patients. The sequence for this protein is disclosed at https://go.drugbank.com under accession number: DB00024.

Antihemophilic Factor, as disclosed in patents CA2124690, CA1339477, is Human recombinant antihemophilic factor or Factor VIII of 2332 residues(glycosylated) is produced by CHO cells. The sequence for this protein is disclosed at https://go.drugbank.com under accession number: DB09329.

Anistreplase, is Human tissue plasminogen activator, purified, glycosylated, 527 residues purified from CHO cells. Eminase is a lyophilized (freeze-dried) formulation of anistreplase, the p-anisoyl derivative of the primary Lys-plasminogen-streptokinase activator complex (a complex of Lys-plasminogen and streptokinase). A p-anisoyl group is chemically conjugated to a complex of bacterial-derived streptokinase and human Plasma-derived Lys-plasminogen proteins. The sequence for this protein is disclosed at https://go.drugbank.com under accession number: DB00029.

Tenecteplase, as disclosed in patents CA2129660, CA1341432, Tenecteplase(527 amino acid) is a glycoprotein developed by introducing the following modifications to the complementary DNA for natural human tPA: a substitution of threonine 103 with asparagine, and a substitution of asparagine 117 with glutamine, both within the kringle 1 domain, and a tetra-alanine substitution at amino acids 296-299 in the protease domain. The sequence for this protein is disclosed at https://go.drugbank.com under accession number: DB00031.

Recombinant human coagulation Factor VIia is intended for promoting hemostasis by activating the extrinsic pathway of the coagulation cascade. NovoSeven is a vitamin K-dependent glycoprotein consisting of 406 amino acid residues, cloned and expressed in hamster kidney cells, the protein is catalytically active in a two-chain form. The sequence for this protein is disclosed at https://go.drugbank.com under accession number: DB00036.

Omalizumab, as disclosed in patents CA2113813, CA1340233, is a recombinant DNA-derived humanized IgG1k monoclonal antibody that selectively binds to human immunoglobulin E. Xolair is produced by a Chinese hamster ovary cell suspension culture in a nutrient medium containing the antibiotic gentamicin. The sequence for this protein is disclosed at https://go.drugbank.com under accession number: DB00043.

Adalimumab is disclosed in patent CA2243459. Adalimumab (1330 amino acids, molecular weight of approximately 148 kilodaltons) is a human monoclonal antibody against TNF-alpha. It is produced by recombinant DNA technology using a mammalian cell expression system. The sequence for this protein is disclosed at https://go.drugbank.com under accession number: DB00051.

Imiglucerase, as disclosed in U.S. Pat. No. 5,549,892, is Human Beta-glucocerebrosidase or Beta-D-glucosyl-N-acylsphingosine glucohydrolase E.C. 3.2.1.45. 497 residue protein with N-linked carbohydrates, MW=59.3 kD. Alglucerase is prepared by modification of the oligosaccharide chains of human Beta-glucocerebros. The sequence for this protein is disclosed at https://go.drugbank.com under accession number: DB00053

Abciximab, as disclosed in patent CA1341357, Abciximab is a Fab fragment of the chimeric human-murine monoclonal antibody 7E3. Abciximab binds to the glycoprotein (GP) IIb/IIIa receptor of human platelets and inhibits platelet aggregation by preventing the binding of fibrinogen, von Willebrand factor, and other adhesive molecules. It also binds to vitronectin (Î±vÎ_3) receptor found on platelets and vessel wall endothelial and smooth muscle cells. The sequence for this protein is disclosed at https://go.drugbank.com under accession number: DB00054.

Abciximab is a Fab fragment of the chimeric human-murine monoclonal antibody 7E3. Abciximab binds to the glycoprotein (GP) IIb/IIIa receptor of human platelets and inhibits platelet aggregation by preventing the binding of fibrinogen, von Willebrand factor, and other adhesive molecules. It also binds to vitronectin (Î±vÎ_3) receptor found on platelets and vessel wall endothelial and smooth muscle cells. The sequence for this protein is disclosed at https://go.drugbank.com under accession number: DB00054.

Interferon beta-1a, as disclosed in patent CA1341604, Human interferon beta (166 residues, glycosylated, MW=22.5 kD) is produced by mammalian cells (Chinese Hamster Ovary cells) into which the human interferon beta gene has been introduced. The amino acid sequence of Avonex is identical to that of natural human interferon beta. The sequence for this protein is disclosed at https://go.drugbank.com under accession number: DB00060.

Infliximab, as disclosed in patent CA2106299, is Tumor necrosis factor (TNF-alpha) binding antibody (chimeric IgG1). It is composed of human constant and murine variable regions. Infliximab is produced by a recombinant cell line cultured by continuous perfusion. The sequence for this protein is disclosed at https://go.drugbank.com under accession number: DB00065.

Follitropin beta, as disclosed in U.S. Pat. Nos. 7,741,268, 5,270,057, CA2037884, is a human follicle stimulating hormone (FSH) preparation of recombinant DNA origin, which consists of two non-covalently linked, non-identical glycoproteins designated as the alpha- and beta-subunits. The alpha- and beta-subunits have 92 and 111 amino acids. The alpha subunit is glycosylated at Asn 51 and Asn 78 while the beta subunit is glycosylated at Asn 7 and Asn 24. Follitropin beta is produced in genetically engineered Chinese hamster cell lines (CHO). The nomenclature â€œbetaâ€ù differentiates it from another recombinant human FSH product that was marketed earlier as follitropin alpha. Follitropin is important in the development of follicles produced by the ovaries. Given by subcutaneous injection, it is used in combination with human chorionic gonadotropin (hCG) to assist in ovulation and fertility. Follitropin may also be used to cause the ovary to produce several follicles, which can then be harvested for use in gamete intrafallopian transfer (GIFT) or in vitro fertilization (IVF). Numerous physio-chemical tests and bioassays indicate that follitropin beta and follitropin alpha are indistinguishable. However, a more recent study showed there is may be a slight clinical difference, with the alpha form tending towards a higher pregnancy rate and the beta form tending towards a lower pregnancy rate, but with significantly higher estradiol (E2) levels. Structural analysis shows that the amino acid sequence of follitropin beta is identical to that of natural human follicle stimulating hormone (FSH). Further, the ogliosaccharide side chains are very similar, but not completely identical to that of natural FSH. However, these small differences do not affect the bioactivity compared to natural FSH. The sequence for this protein is disclosed at https://go.drugbank.com under accession number: DB00066.

Trastuzumab, as disclosed in patent CA2103059, A recombinant IgG1 kappa, humanized monoclonal antibody that selectively binds with high affinity in a cell-based assay (Kd=5 nM) to the extracellular domain of the human epidermal growth factor receptor protein. Produced in CHO cell culture. The sequence for this protein is disclosed at https://go.drugbank.com under accession number: DB00072.

Rituximab, as disclosed in patents CA2149329, CA1336826, U.S. Pat. No. 5,736,137, is a genetically engineered chimeric murine/human monoclonal antibody directed against the CD20 antigen found on the surface of normal and malignant B lymphocytes. The antibody is an IgG1 kappa immunoglobulin containing murine light- and heavy-chain variable region sequences and human constant region sequences. Rituximab is composed of two heavy chains of 451 amino acids and two light chains of 213 amino acids. The sequence for this protein is disclosed at https://go.drugbank.com under accession number: DB00073.

Basiliximab, as disclosed in patent CA2038279, is a recombinant chimeric (murine/human) monoclonal antibody (IgG1k) that functions as an immunosuppressive agent, specifically binding to and blocking the interleukin-2 receptor a-chain (IL-2R alpha, also known as CD25 antigen) on the surface of activated T-lymphocytes. It is a 144 kDa glycoprotein obtained from fermentation of an established mouse myeloma cell line genetically engineered to express plasmids containing the human heavy and light chain constant region genes and mouse heavy and light chain variable region genes encoding the RFT5 antibody that binds selectively to the IL-2R alpha. The sequence for this protein is disclosed at https://go.drugbank.com under accession number: DB00074.

Muromonab is a Murine monoclonal antibody specific to CD3 T-cell lymphocyte antigens. More specifically it is a purified murine (mouse) monoclonal antibody, directed against the CD3 (T3) receptor on the surface of human T-cells (T-lymphocytes) cultured using the murine ascites method. Muromonab is 93% monomeric immune globulin G type 2a (IgG2a). The sequence for this protein is disclosed at https://go.drugbank.com under accession number: DB00075.

Ibritumomab, as disclosed in patent CA2149329, is Indium conjugated murine IgG1 kappa monoclonal antibody directed against the CD20 antigen, which is found on the surface of normal and malignant B lymphocytes. Ibritumomab is produced in Chinese hamster ovary cells and is composed of two murine gamma 1 heavy chains of 445 amino acids each and two kappa light chains of 213 amino acids each. The sequence for this protein is disclosed at https://go.drugbank.com under accession number: DB00078.

Tositumomab is Murine IgG2a lambda monoclonal antibody against CD20 antigen (2 heavy chains of 451 residues, 2 lambda chains of 220 residues). It is produced in an antibiotic-free culture of mammalian cells. It can be covalently linked to Iodine 131 (a radioactive isotope of iodine). The sequence for this protein is disclosed at https://go.drugbank.com under accession number: DB00081.

Alemtuzumab, as disclosed in patent CA1339198, is a Humanized monoclonal antibody specific to lymphocyte antigens. It is a recombinant DNA-derived humanized monoclonal antibody (Campath-1H) that is directed against the 21-28 kD cell surface glycoprotein, CD52. The Campath-1H antibody is an IgG1 kappa with human variable framework and constant regions, and complementarity-determining regions from a murine (rat) monoclonal antibody (Campath-1G). Campath is produced in mammalian cell (Chinese hamster ovary) suspension culture in a medium containing neomycin. The sequence for this protein is disclosed at https://go.drugbank.com under accession number: DB00087.

Laronidase is a Human recombinant alpha-L-iduronidase, produced by recombinant DNAtechnology in a Chinese hamster ovary cell line. Laronidase is a glycoprotein with a predicted amino acid sequence of the recombinant form, as well as the nucleotide sequence that encodes it, are identical to a polymorphic form of human a-L-iduronidase. It contains 6 N-linked oligosaccharide modification sites. The sequence for this protein is disclosed at https://go.drugbank.com under accession number: DB00090.

Efalizumab is Humanized IgG1 kappa isotype monoclonal antibody that binds to human CD11a. It is produced in a Chinese hamster ovary mammalian cell expression system in a nutrient medium containing the antibiotic gentamicin. The sequence for this protein is disclosed at https://go.drugbank.com under accession number: DB00095.

Choriogonadotropin alfa, as disclosed in U.S. Pat. Nos. 6,706,681, 5,767,251, is Recombinant human chorionic gonadotropin with a 92-residue alpha subunit and a 145 residue beta subunit. Glycosylation consists of N- and O-linked carbohydrate moieties linked to N-52 and N-78 (on alpha subunit) and N13 and 30, 5121, 127, 132 and 138 (on beta subunit). The primary structure of the alpha-chain of r-hCG is identical to that of the alpha-chain of hCG, FSH and LH. The sequence for this protein is disclosed at https://go.drugbank.com under accession number: DB00097.

Coagulation Factor IX (Recombinant) is a glycoprotein with an approximate molecular mass of 55, 000 Da consisting of single chain of 415 amino acids. The Sequence is identical to the A148 allelic form of plasma-derived factor IX. The sequence for this protein is disclosed at https://go.drugbank.com under accession number: DB13152.

Agalsidase beta, as disclosed in patent CA2265464, is recombinant human alpha-galactosidase-A produced in CHO cells. The mature protein comprises 2 subunits of 398 residues. The sequence for this protein is disclosed at https://go.drugbank.com under accession number: DB00103.

Palivizumab, as disclosed in patent CA2197684, is a humanized, recombinant, monoclonal antibody (IgG1k) directed agaisnt the epitope in the A antigenic site of the F protein of respiratory syncytial virus (RSV). Synagis is a composite of human (95%) and murine (5%) antibody sequences. The human heavy chain sequence is derived from the constant domains of human IgG1 and the variable framework regions of the VH genes Cor (1) and Cess (2). The human light chain sequence is derived from the constant domain of Ck and the variable framework regions of the VL gene K104 withJk-4. Palivizumab is expressed from a stable murine myeloma cell line (NS0). Palivizumab is composed of to heavy chains (50.6 kDa each) and two light chains (27.6 kDa each), contains 1-2% carbohydrate by weight and has a molecular weight of 147.7 kDa±1 kDa (MALDI-TOF). The sequence for this protein is disclosed at https://go.drugbank.com under accession number: DB00110.

Daclizumab is Humanized, recombinant, monoclonal antibody (IgG1k) directed against the epitope in the A antigenic site of the F protein of respiratory syncytial virus (RSV). Synagis is a composite of human (95%) and murine (5%) antibody sequences. The human heavy chain sequence is derived from the constant domains of human IgG1 and the variable framework regions of the VH genes Cor (1) and Cess (2). The human light chain sequence is derived from the constant domain of Ck and the variable framework regions of the VL gene K104 withJk-4. Palivizumab is expressed from a stable murine myeloma cell line (NS0). Palivizumab is composed of to heavy chains (50.6 kDa each) and two light chains (27.6 kDa each), contains 1-2% carbohydrate by weight and has a molecular weight of 147.7 kDa±1 kDa (MALDI-TOF). The sequence for this protein is disclosed at https://go.drugbank.com under accession number: DB00111.

Bevacizumab, as disclosed in patent CA2286330, CA2145985, is recombinant (derived from CHO-gentamycin), humanized, monoclonal IgG1 antibody. It Inhibits the biologic activity of human vascular endothelial growth factor (VEGF) by binding to it. Comprises human framework regions and murine complementarity-determining regions. The sequence for this protein is disclosed at https://go.drugbank.com under accession number: DB00112.

Arcitumomab, as disclosed in U.S. Pat. Nos. 8,420,081, 7,790,142, 8,226,949, is a reduced Fab fragment of the murine IgG1 monoclonal antibody IMMU-4 (also called NP-4) with specificity for carcinoembryonic antigen. Covalently labeled with Technitium 99. The sequence for this protein is disclosed at https://go.drugbank.com under accession number: DB17201.

Soliris, as disclosed in patent CA2189015, is a formulation of eculizumab which is a recombinant humanized monoclonal IgG2/4; K antibody. Recombinant; produced in murine myeloma cell culture. Eculizumab contains human constant regions from human IgG2 sequences and human IgG4 sequences and murine complementarity-determining regions grafted onto the human framework light- and heavy-chain variable regions and has a molecular weight of approximately 148 kDa. The sequence for this protein is disclosed at https://go.drugbank.com under accession number: DB01257.

Panitumumab (ABX-EGF) is an anti-neoplastic agent. It is a recombinant human IgG2 monoclonal antibody that binds specifically to the human epidermal growth factor receptor (EGFR). The sequence for this protein is disclosed at https://go.drugbank.com under accession number: DB01269.

Idursulfase is a recombinant (from human cell line) form of human lysosomal enzyme, iduronate-2-sulfatase. Idursulfase is a 525-amino acid glycoprotein, which contains 8 N-linked glycosylation sites, occupied by complex oligosaccharide structures. Its enzyme activity depends on the post-translational modification of a specific cysteine to formylglycine. The sequence for this protein is disclosed at https://go.drugbank.com under accession number: DB01271.

Alglucosidase alfa, as disclosed in patent CA2416492, is a recombinant (CHO cell derived) form of the human lysosomal enzyme, acid alpha-glucosidase (GAA), which is essential for the degradation of glygogen to glucose. It hydrolyses the alfa-1, 4- and alfa-1, 6-glycosidic linkages of lysosomal glycogen. The mature polypeptide is 883 residue long with a mass of 98, 008 daltons and a total mass of approximately 109, 000 daltons of the full-length glycosylated protein, The sequence for this protein is disclosed at https://go.drugbank.com under accession number: DB01272.

Galsulfase is a recombinant variant form of the polymorphic human enzyme, N-acetylgalactosamine 4-sulfatase. It is a glycoprotein with a molecular weight of approximately 56 kD and comprises 495 amino acids. It contains 6 N-linked glycosylation sites, four of which carry a bis-mannose-6-phosphate manose7 oligosaccharide for specific cellular recognition. It requires Ca-formylglycine for its catalytic activity. This residue is a post-translational modification of Cys53 and conserved in all members of the sulfatase enzyme family. The sequence for this protein is disclosed at https://go.drugbank.com under accession number: DB01279.

Abatacept, as disclosed in patent CA2110518, is recombinant (CHO cell derived), soluble fusion protein that links the extracellular domain of human cytotoxic T-lymphocyte-associated antigen 4 (CTLA-4), to the modified Fc (hinge, CH2, and CH3 domains) portion of human IgG1. Abatacept is a glycosylated fusion protein with molecular weight of 92, 300 Da and it is a homodimer of two polypeptide chains of 357 amino acids. The drug has activity as a selective co-stimulation modulator with inhibitory activity on T lymphocytes. The sequence for this protein is disclosed at https://go.drugbank.com under accession number: DB01281.

Canakinumab is a recombinant (from murine Sp2/0-Ag14 cell line), human anti-human-IL-1_ monoclonal antibody that belongs to the IgG1/isotype subclass. It comprises of two 447- or 448-residue heavy chains and two 214-residue light chains. Both heavy chains of canakinumab contain oligosaccharide chains linked to the protein backbone at N298. It binds to human IL-1_, thereby neutralizing its inflammatory activity by preventing its interaction with IL-1 receptors. However it does not bind IL-1alpha or IL-1 receptor antagonist (IL-1ra). It is marketed under the brand name Ilaris. The sequence for this protein is disclosed at https://go.drugbank.com under accession number: DB06168.

Ipilimumab, as disclosed in patent CA2381770, is a recombinant, human monoclonal IgG1 kappa immunoglobin. It is an antineoplastic agent developed by Bristol-Myers Squibb and Medarex for the treatment of unresectable or metastatic melanoma in adults. Ipilimumab received FDA approval on Mar. 25, 2011. The sequence for this protein is disclosed at https://go.drugbank.com under accession number: DB06186.

Tocilizumab, as disclosed in patents CA2201781, CA1341152, is a recombinant, humanized, anti-human interleukin 6 receptor (IL-6R) monoclonal antibody. The light chain is made up of 214 amino acids and the heavy chain is made up of 448 amino acids. The sequence for this protein is disclosed at https://go.drugbank.com under accession number: DB06273.

Pertuzumab, as disclosed in patents CA2376596, CA2579861, is a recombinant, humanized monoclonal antibody that targets the extracellular dimerization domain (Subdomain II) of the human epidermal growth factor receptor 2 protein (HER2). Two heavy chains and two lights chains are composed of 448 and 214 residues respectively. FDA approved Jun. 8, 2012. The sequence for this protein is disclosed at https://go.drugbank.com under accession number: DB06366.

Rilonacept, as disclosed in U.S. Pat. Nos. 5,844,099, 8,114,394, 8,080,248, Dimeric fusion protein consisting of portions of IL-1R and the IL-1R accessory protein, linked to the Fc portion of immunoglobulin G1. It inhibits interleukin 1 and is used in the treatment of cryopyrin-associated periodic syndromes (CAPS), in adults and children oven than 12 of ageold. The sequence for this protein is disclosed at https://go.drugbank.com under accession number: DB06372.

Denosumab, as disclosed in patents CA2257247, CA2274987, CA2285746, CA2400929, CA2328140, is a fully human IgG2 monoclonal antibody, specific to receptor activator of nuclear factor kappa-B ligand (RANKL). It suppresses bone resorption markers in patients suffering from metastatic tumors and is being investigated in multiple clinical trials for the prevention and treatment of bone metastases. Each light chain consists of 215 amino acids and the heavy chain consists of 448 amino acids. FDA approved on Jun. 1, 2010. The sequence for this protein is disclosed at https://go.drugbank.com under accession number: DB06643.

Belatacept is a recombinant (CHO cells derived) soluble fusion protein, which links the extracellular domain of human cytotoxic CTLA-4 to the modified Fc portion of human IgG1, thereby selectively blocking the process of T-cell activation. It is a glycosylated fusion protein, which is a homodimer of two homologous polypeptide chains of 357 amino acids each. The drug acts as a selective co-stimulation modulator with inhibitory activity on T lymphocytes. It differs from abatacept (Orencia) by only 2 amino acids. It is approved for the treatment of rheumatoid arthritis. It was developed by Bristol-Myers-Squibb. FDA approved on Jun. 15, 2011. The sequence for this protein is disclosed at https://go.drugbank.com under accession number: DB06681.

Velaglucerase alfa, as disclosed in U.S. Pat. No. 7,138,262, is a gene-activated human recombinant glucocerebrosidase. It is used to treat Type 1 Gaucher disease, caused by a deficiency of the lysosomal enzyme glucocerebrosidase. Additionally, Velaglucerase alfa has also been investigated for use in Type 3 Gaucher disease. The sequence for this protein is disclosed at https://go.drugbank.com under accession number: DB06720.

Brentuximag vedotin or Adcetris is an antibody-drug conjugate that combines an anti-CD30 antibody and the drug monomethyl auristatin E (MMAE). It is an anti-cancer drug used to treat Hodgkin lymphoma and systemic anaplastic large cell lymphoma. It was approved in 2011 but in January 2012, the drug label was revised to include a boxed warning of progressive multifocal leukoencephalopathy and death following JC virus infection. The sequence for this protein is disclosed at https://go.drugbank.com under accession number: DB08870.

Taliglucerase alfa is a recombinant human glucocerebrosidase (a lysosomal enzyme). Elelyso used in patients with type 1 Gaucher's disease, The sequence for this protein is disclosed at https://go.drugbank.com under accession number: DB08876.

RAV12 is a monoclonal antibody that is being studied in the treatment of certain cancers. It binds to a carbohydrate molecule found on gastric, colon, pancreatic, prostate, ovarian, breast, and kidney cancer cells. Administering RAV12 along with gemcitabine may kill more tumor cells than either one alone. The sequence for this protein is disclosed at https://go.drugbank.com under accession number: DB08879.

Aflibercept, as disclosed in U.S. Pat. Nos. 7,306,799, 7,531,173, 7,374,758, 7,608,261, 7,070,959, 7,374,757, is a vascular endothelial growth factor (VEGF) inhibitor. It is a recombinant dimeric fusion glycoprotein that comprises (VEGF) binding portions from the extracellular domains of human VEGF receptors 1 and 2, this is fused to the Fc portion of human IgG1. It contains approximately 15% glycosylation to give a total molecular weight of 115 kDa (protein part=96.9 kDa). It has 5 putative N-glycosylation sites on each polypeptide chain and the attached carbohydrates exhibit some degree of chain heterogeneity, including heterogeneity in terminal sialic acid residues, except at the site associated with the Fc domain, which is unsialylated. The sequence for this protein is disclosed at https://go.drugbank.com under accession number: DB08885.

Raxibacumab is a recombinant (murine cell line derived), human IgG1 monoclonal antibody that binds the protective antigen (PA) component of B. anthracis toxin. FDA approved on Dec. 14, 2012. The sequence for this protein is disclosed at https://go.drugbank.com under accession number: DB08902.

Obinutuzumab, as disclosed in patent N.A. Humanized monoclonal antibody used along with chlorambucil for the treatment of chronic lymphocytic leukemia. It was approved by the FDA in November 2013 and is marketed under the brand name Gazyva. It carries a black box warning of fatal Hepatitis B Virus (HBV) reactivation and fatal Progressive Multifocal Leukoencephalopathy (PML). The sequence for this protein is disclosed at https://go.drugbank.com under accession number: DB08935.

Follitropin alpha is a recombinant (CHO cell derived) human follicle stimulating hormone (FSH). It consists of two non-covalently linked, non-identical glycoproteins designated as the alpha (92 amino acids)- and beta (111 amino acids)- subunits. The alpha subunit is glycosylated at N51 and N78 while the beta subunit is glycosylated at N7 and N24. Follitropin alpha was the world's first recombinant human FSH preparation. The term alpha differentiates it from another recombinant human FSH product that was marketed later as follitropin beta. Follitropin is important in the development of follicles produced by the ovaries. Given by subcutaneous injection, it is used in combination with human chorionic gonadotropin (hCG) to assist in ovulation and fertility. Follitropin may also be used to cause the ovary to produce several follicles, which can then be harvested for use in gamete intrafallopian transfer (GIFT) or in vitro fertilization (IVF). Numerous physio-chemical tests and bioassays indicate that follitropin beta and follitropin alpha are indistinguishable. However, a more recent study showed there is may be a slight clinical difference, with the alpha form tending towards a higher pregnancy rate than beta but with significantly higher estradiol (E2) levels. The amino acid sequence of follitropin beta is identical to that of natural human follicle stimulating hormone (FSH). Further, the ogliosaccharide side-chains are very similar, but not completely identical to that of natural FSH. However, these small differences do not affect the bioactivity compared to natural FSH. The sequence for this protein is disclosed at https://go.drugbank.com under accession number: DB00066.

Romiplostim is a thrombopoiesis stimulating dimer Fc-peptide fusion protein (peptibody) to increase platelet production through activation of the thrombopoietin receptor. The peptibody molecule has two identical single-chain subunits, each one is made up of 269 amino acid residues. Each subunit consists of an IgG1 Fc carrier domain that is covalently attached to a polypeptide sequence that contains two binding domains to interact with thrombopoietin receptor c-Mpl. Each domain consists of 14 amino acids. Interestingly, romiplostim's amino acid sequence is not similar to that of endogenous thrombopoietin. Romiplostim is produced by recombinant DNA technology in Escherichia coli. FDA approved on Aug. 22, 2008. The sequence for this protein is disclosed at https://go.drugbank.com under accession number: DB05332.

Natalizumab is a humanized IgG4k monoclonal antibody produced in murine myeloma cells. Natalizumab contains human framework regions and the complementarity-determining regions of a murine antibody that binds to a4-integrin. Natalizumab was voluntarily withdrawn from U.S. market because of risk of Progressive multifocal leukoencephalopathy (PML). It was returned to market July, 2006. The sequence for this protein is disclosed at https://go.drugbank.com under accession number: DB00108.

Secukinumab, as disclosed in patent US20130202610, is a human monoclonal antibody, secukinumab (cosentyx) was designed for the treatment of uveitis, rheumatoid arthritis, ankylosing spondylitis, and psoriasis. Secukinumab is an interleukin-17A inhibitor marketed by Novartis. On Jan. 19, 2015, secukinumab was approved by the European Commission as a first line systemic treatment in moderate to severe adult plaque psoriasis. On Jan. 21, 2015, the United States Food and Drug Administration announced that it had approved secukinumab to treat adults with moderate-to-severe plaque psoriasis. The sequence for this protein is disclosed at https://go.drugbank.com under accession number: DB09029.

Drotrecogin alfa, as disclosed in patents CA2036894, CA2139468, is activated human protein C that is synthesized by recombinant DNA technology. It is a glycoprotein of approximately 55 kilodalton molecular weight, consisting of a heavy chain and a light chain linked by a disulfide bond. Drotrecogin alfa was withdrawn from the market after a major study indicated that it was not effective in improving outcomes in patients with sepsis. The sequence for this protein is disclosed at https://go.drugbank.com under accession number: DB00055.

Alefacept is an immunosuppressive dimeric fusion protein that consists of the extracellular CD2-binding portion of the human leukocyte function antigen-3 (LFA-3) linked to the Fc (hinge, CH2 and CH3 domains) portion of human IgG1. Produced by CHO cells, mW is 91.4 kD. The sequence for this protein is disclosed at https://go.drugbank.com under accession number: DB00092.

Urokinase, as disclosed in U.S. Pat. No. 4,258,030, is a low molecular weight form of human urokinase, that consists of an A chain of 2, 000 daltons linked by a sulfhydryl bond to a B chain of 30, 400 daltons. Recombinant urokinase plasminogen activator, The sequence for this protein is disclosed at https://go.drugbank.com under accession number: DB00013.

Gemtuzumab ozogamicin, as disclosed in U.S. Pat. Nos. 5,585,089, 5,773,001, is a recombinant humanized IgG4, kappa antibody conjugated with a cytotoxic antitumor antibiotic, calicheamicin, isolated from fermentation of a bacterium, Micromonospora echinospora ssp. calichensis. The antibody portion of Mylotarg binds specifically to the CD33 antigen, The anti-CD33 hP67.6 antibody is produced by mammalian cell suspension culture using a myeloma NS0 cell line. The sequence for this protein is disclosed at https://go.drugbank.com under accession number: DB00056.

Satumomab Pendetide is tumor associated glycoprotein (TAG) 72 (B72.3) monoclonal antibody conjugated with Indium 111 for radioimaging colon tumors. Satumomab Pendetide (trade name: OncoScint) is no longer commercially available. The sequence for this protein is disclosed at https://go.drugbank.com under accession number: DB00057.

Alirocumab is a biopharmaceutical drug approved by the FDA in July 2015 as a second line treatment for high cholesterol for adults whose LDL-cholesterol (LDL-C) is not controlled by diet and statin treatment. It is a human monoclonal antibody administered by subcutaneous injection that belongs to a novel class of anti-cholesterol drugs, known as PCSK9 inhibitors, and it was the first such agent to receive FDA approval. The FDA approval was contingent on the completion of further clinical trials to better determine efficacy and safety. PCSK9 inhibition facilitates more LDL-C clearance from the blood. The sequence for this protein is disclosed at https://go.drugbank.com under accession number: DB09302.

Ancestim is a recombinant methionyl human stem cell factor, branded by Amgen as StemGen. It was developed by Amgen and sold to Biovitrium, now Swedish Orphan Biovitrum, in December, 2008. It is a 166 amino acid protein produced by E. coli bacteria into which a gene has been inserted for soluble human stem cell factor. It has a monomeric molecular weight of approximately 18, 500 daltons and normally exists as a noncovalently associated dimer. The protein has an amino acid sequence that is identical to the natural sequence predicted from human DNA sequence analysis, except for the addition of an N-terminal methionine retained after expression in E. coli. Because Ancestim is produced in E. coli, it is nonglycosylated. Ancestim is supplied as a sterile, white, preservative-free, lyophilised powder for reconstitution and administration as a subcutaneous (SC) injection and is indicated for use in combination with filgrastim for mobilizing peripheral hematopoietic stem cells for later transplanation in certain cancer patients, The sequence for this protein is disclosed at https://go.drugbank.com under accession number: DB09103.

Recombinant Antithrombin Alfa, contains six cysteine residues forming three disulphide bridges and 3-4 N-linked carbohydrate moieties. The glycosylation profile of antithrombin (Recombinant) is different from plasma-derived antithrombin, which results in an increased heparin affinity. When assayed in the presence of excess of heparin the potency of the recombinant product is not different from that of plasma-derived product. The sequence for this protein is disclosed at https://go.drugbank.com under accession number: DB11166.

Antithrombin III human is a plasma alpha 2 glycoprotein that accounts for the major antithrombin activity of normal plasma and also inhibits several other enzymes. It is a member of the serpin superfamily. The sequence for this protein is disclosed at https://go.drugbank.com under accession number: DB11598.

Asfotase Alfa, as disclosed in patent NA, Asfotase Alfa is a first-in-class bone-targeted enzyme replacement therapy designed to address the underlying cause of hypophosphatasia (HPP)—deficient alkaline phosphatase (ALP). Hypophosphatasia is almost always fatal when severe skeletal disease is obvious at birth. By replacing deficient ALP, treatment with Asfotase Alfa aims to improve the elevated enzyme substrate levels and improve the body's ability to mineralize bone, thereby preventing serious skeletal and systemic patient morbidity and premature death. Asfotase alfa was first approved by Pharmaceuticals and Medicals Devices Agency of Japan (PMDA) on Jul. 3, 2015, then approved by the European Medicine Agency (EMA) on Aug. 28, 2015, and was approved by the U.S. Food and Drug Administration (FDA) on Oct. 23, 2015. The sequence for this protein is disclosed at https://go.drugbank.com under accession number: DB09105.

Atezolizumab, as disclosed in patent NA, Atezolizumab is an Fc-engineered, humanized, monoclonal antibody that binds to PD-L1 and blocks interactions with the PD-1 and B7.1 receptors. Atezolizumab is a non-glycosylated IgG1 kappa immunoglobulin that has a calculated molecular mass of 145 kDa. Atezolizumab was approved in the US in May, 2016 under the brand name Tecentriq. The sequence for this protein is disclosed at https://go.drugbank.com under accession number: DB11595.

Blinatumomab, as disclosed in patent US20120328618, US20130323247, U.S. Pat. Nos. 7,235,641, 7,575,923, 7,635,472, 8,247,194, Blinatumomab is a BiTE-class (bi-specific T-cell engagers) constructed monoclonal antibody indicated for the treatment of Philadelphia chromosome-negative relapsed or refractory B-cell precursor acute lymphoblastic leukemia (ALL). Blinatumomab is manufactured by Amgen Inc. and marketed under the brand Blincyto™. A full treatment regimen consisting of two cycles of four weeks each, is priced at $178 000 USD. Blinatumomab was approved in December 2014 under the FDA's accelerated approval program, which allows approval of a drug to treat a serious or life-threatening disease based on clinical data showing the drug has an effect on a surrogate endpoint reasonably likely to predict clinical benefit to patients. The sequence for this protein is disclosed at https://go.drugbank.com under accession number: DB09052.

C1 Esterase Inhibitor (Human), as disclosed in patent NA, Recombinant human C1 esterase inhibitor is a human protein developed through Pharming's proprietary technology where the human protein is expressed in milk of transgenic rabbits. Hereditary Angioedema (HAE) is a human genetic disorder caused by a shortage of C1 inhibitor activity and results in an overreaction of the immune system. The disease is characterized by acute attacks of painful and in some cases fatal swelling of several soft tissues (edema), which may last up to five days when untreated. The sequence for this protein is disclosed at https://go.drugbank.com under accession number: DB06404.

Coagulation Factor XIII A-Subunit (Recombinant) is a recombinant human factor XIII-A2 homodimer composed of two factor XIII (FXIII) A-subunits. The FXIII A-subunit is a 731 amino acid chain with an acetylated N-terminal serine. When FXIII is activated by thrombin, a 37 amino acid peptide is cleaved from the N-terminus of the A-subunit. Coagulation Factor XIII A-Subunit (Recombinant) is manufactured as an intracellular, soluble protein in yeast (Saccharomyces cerevisiae) production strain containing the episomal expression vector, pD16. It is subsequently isolated by homogenization of cells and purification by several chromatography steps, including hydrophobic interaction and ion exchange chromatography. No human or animal derived products are used in the manufacturing process. The sequence for this protein is disclosed at https://go.drugbank.com under accession number: DB09310.

Conestat alfa is a recombinant, human C1-inhibitor (rhC 1INH), for the treatment of acute attacks of hereditary angioedema (HAE) due to C1 esterase inhibitor deficiency in adults. Conestat alfa was approved in October 2010 in all 27 EU member states plus Norway, Iceland and Liechtenstein. The sequence for this protein is disclosed at https://go.drugbank.com under accession number: DB09228.

Daratumumab is an anti-cancer drug indicated for multiple myeloma in patients who have received at least 3 prior treatments. It was granted accelerated approval by the FDA in November 2016. Marketed under the brand name Darzalex by Janssen Biotech, daratumumab is the first monoclonal antibody injection approved for this indication and provides another options for patients with multiple myeloma resistant to other therapies. Daratumumab induces apoptosis of cancer cells by targeting the CD38 epitope, which is highly expressed on haematological malignancies. The sequence for this protein is disclosed at https://go.drugbank.com under accession number: DB09331.

Elotuzumab, as disclosed in patent US2014055370, is a humanized IgG1 (Immunoglobulin G) monoclonal antibody indicated in combination with lenalidomide and dexamethasone for the treatment of patients with multiple myeloma who have received one to three prior therapies. Elotuzumab targets SLAMF7, also known as Signaling Lymphocytic Activation Molecule Family member 7, a cell surface glycoprotein. Elotuzumab consists of the complementary determining regions (CDR) of the mouse antibody, MuLuc63, grafted onto human IgG1 heavy and kappa light chain frameworks. Elotuzumab is produced in NS0 cells by recombinant DNA technology. Elotuzumab has a theoretical mass of 148.1 kDa for the intact antibody. Elotuzumab was approved on Nov. 30, 2015 by the U.S. Food and Drug Administration. Elotuzumab is marketed under the brand Empliciti™ by Bristol-Myers Squibb. The sequence for this protein is disclosed at https://go.drugbank.com under accession number: DB06317.

Evolocumab is a monoclonal antibody designed for the treatment of hyperlipidemia by Amgen. It is a subcutaneous injection approved by the FDA for individuals on maximum statin therapy who still require additional LDL-cholesterol lowering. It is approved for both homozygous and heterozygous familial cholesterolemia as an adjunct to other first-line therapies. Evolocumab is a human IgG2 monoclonal antibody that inhibits proprotein convertase subtilisin/kexin type 9 (PCSK9). PCSK9 is a protein that targets LDL receptors for degradation, therefore reducing the liver's ability to remove LDL-cholesterol (LDL-C), or bad cholesterol, from the blood. Evolocumab is designed to bind to PCSK9 and inhibit PCSK9 from binding to LDL receptors on the liver surface, resulting in more LDL receptors on the surface of the liver to remove LDL-C from the blood. Evolocumab is the second PCSK9 inhibitor on the market, first being alirocumab. The sequence for this protein is disclosed at https://go.drugbank.com under accession number: DB09303.

Hyaluronidase (Human Recombinant), as disclosed in U.S. Pat. No. 7,767,429, is a purified preparation of the enzyme recombinant human hyaluronidase. Hyaluronidase (Human Recombinant) is produced by genetically engineered Chinese Hamster Ovary (CHO) cells containing a DNA plasmid encoding for a soluble fragment of human hyaluronidase (PH20). The purified hyaluronidase glycoprotein contains 447 amino acids with an approximate molecular weight of 61, 000 Daltons. The sequence for this protein is disclosed at https://go.drugbank.com under accession number: DB06205.

Idarucizumab, sold under the brandname Praxbind, is a humanized monoclonal antibody fragment (Fab) derived from an IgG1 isotype molecule, whose target is the direct thrombin inhibitor dabigatran. Using recombinant expression technology, idarucizumab is produced in a well characterized recombinant (mammalian) CHO cell line and is purified using standard technology. Idarucizumab is composed of a light chain of 219 amino acids and a heavy chain fragment of 225 amino acids, covalently linked together by one disulfide bond between cysteine 225 of the heavy chain fragment and cysteine 219 of the light chain, and has an estimated molecular mass of approximately 47, 766 Daltons. The sequence for this protein is disclosed at https://go.drugbank.com under accession number: DB09264.

Vedolizumab, as disclosed in patent US2012151248, is a recombinant humanized IgG1 monoclonal antibody directed against the human lymphocyte_4_7 integrin, a key mediator of gastrointestinal inflammation. It is used in the treatment of moderate to severe active ulcerative colitis and Crohn's disease for patients who have had an inadequate response with, lost response to, or were intolerant to inhibitors of tumor necrosis factor-alpha (TNF-alpha) or other conventional therapies. By blocking its primary target, _4_7 integrin, vedolizumab reduces inflammation in the gut. The sequence for this protein is disclosed at https://go.drugbank.com under accession number: DB09033.

Ustekinumab is a human immunosuppressive drug ustekinumab developed by the biotechnology company Centocor. It is a laboratory manufactured, monoclonal antibody directed against interleukins IL-12 and IL-23. The sequence for this protein is disclosed at https://go.drugbank.com under accession number: DB05679.

Turoctocog alfa is a recombinant factor VIII (rFVIII) with a truncated B-domain made from the sequence coding for 10 amino acids from the N-terminus and 11 amino acids from the C-terminus of the naturally occurring B-domain. Turoctocog alfa is produced in Chinese hamster ovary (CHO) cells without addition of any human- or animal-derived materials.

During secretion, some rFVIII molecules are cleaved at the C-terminal of the heavy chain (HC) at amino acid 720, and a monoclonal antibody binding C-terminal to this position is used in the purification process allowing isolation of the intact rFVIII. It was first launched in Germany in January 2014 and has been approved in the US, EU and Japan, The sequence for this protein is disclosed at https://go.drugbank.com under accession number: DB09109.

Simoctocog Alfa is a recombinant B-domain deleted (BDD) rFVIII produced in genetically modified human embryonic kidney (HEK) 293F cells. The harvested product is concentrated and purified by a series of chromatography steps. No animal proteins are used in the purification process and no human albumin is used as a stabiliser in the manufacture of Human-c1 rhFVIII. Simoctocog Alfa is a glycoprotein consisting of 1440 amino acids with an approximate molecular mass of 170 kDa, comprising the FVIII domains A1-A2+A3-C1-C2 whereas the B-domain, present in the full-length plasma-derived FVIII, has been deleted and replaced by a 16 amino acid linker. The sequence for this protein is disclosed at https://go.drugbank.com under accession number: DB09108.

Siltuximab, as disclosed in U.S. Pat. No. 7,612,182, is a chimeric (human-mouse) monoclonal immunoglobulin G1-kappa antibody produced in a Chinese hamster ovary (CHO) cell line by recombinant DNA technology. Siltuximab prevents the binding of IL-6 to soluble and membrane-bound IL-6 receptors by forming high affinity complexes with human interleukin-6 (IL-6). Its use is indicated for the treatment of adult patients with multicentric Castleman's disease (MCD) who are human immunodeficiency virus (HIV) negative and human herpesvirus-8 (HHV-8) negative. MCD is a rare blood disorder caused by dysregulated IL-6 production, proliferation of lymphocytes, and subsequent enlargement of the lymph nodes. It is administered as a 1 hour intravenous infusion every 3 weeks. The sequence for this protein is disclosed at https://go.drugbank.com under accession number: DB09036.

Sebelipase alfa is a recombinant form of the enzyme lysosomal acid lipase (LAL) approved for the treatment of lysosomal acid lipase deficiency (LAL-D). The amino acid sequence for sebelipase alfa is the same as the amino acid sequence for human LAL. Sebelipase alfa is an orphan drug which is expected to cost about $310, 000 for annual treatment in the United States. Sebelipase alfa is marketed under the brand name Kanuma™ by Alexion Pharmaceuticals, Inc. The sequence for this protein is disclosed at https://go.drugbank.com under accession number: DB11563.

Ramucirumab, as disclosed in patent US2013067098, is a human monoclonal antibody (IgG1) against vascular endothelial growth factor receptor 2 (VEGFR2), a type II trans-membrane tyrosine kinase receptor expressed on endothelial cells. By binding to VEGFR2, ramucirumab prevents binding of its ligands (VEGF-A, VEGF-C, and VEGF-D), thereby preventing VEGF-stimulated receptor phosphorylation and downstream ligand-induced proliferation, permeability, and migration of human endothelial cells. VEGFR stimulation also mediates downstream signalling required for angiogenesis and is postulated to be heavily involved in cancer progression, making it a highly likely drug target. In contrast to other agents directed against VEGFR-2, ramucirumab binds a specific epitope on the extracellular domain of VEGFR-2, thereby blocking all VEGF ligands from binding to it. Ramucirumab is indicated for us in advanced gastric or gastro-esophageal junction adenocarcinoma as a single agent or in combination with paclitaxel after prior fluoropyrimidine- or platinum-containing chemotherapy. The sequence for this protein is disclosed at https://go.drugbank.com under accession number: DB05578.

Pembrolizumab, as disclosed in patent US2012135408, is an antibody drug that targets the cell surface receptor programmed cell death protein 1 (PD-1) found on T cells. By preventing the binding of its ligands (PD-L1 and PD-L2), pembrolizumab induces an antitumor immune response. Upregulation of PD-1 ligands occurs in some tumors and signaling through this pathway can contribute to inhibition of active T-cell immune surveillance of tumors. Its use is indicated for the treatment of patients with unresectable or metastatic melanoma and disease progression following therapy with ipilimumab and, if BRAF V600 mutation positive, a BRAF inhibitor. Due to its success in clinical trials, pembrolizumab was approved early to allow quick patient access and was given breakthrough therapy and orphan drug designation. Pembrolizumab (as Keytruda) was approved by the U.S. Food and Drug Administration to treat advanced cases of the most common type of lung malignancy, non-small cell lung cancer (NSCLC) on Oct. 2, 2015. The sequence for this protein is disclosed at https://go.drugbank.com under accession number: DB09037.

Peginterferon beta-1a is an interferon beta-1a to which a single, linear 20, 000 dalton (Da) methoxy poly(ethyleneglycol)-O-2-methylpropionaldehyde molecule is covalently attached to the alpha amino group of the N-terminal amino acid residue. The interferon beta-1a portion is produced as a glycosylated protein using genetically-engineered Chinese hamster ovary cells into which the human interferon beta gene has been introduced. The amino acid sequence of the recombinant interferon beta-1a is identical to that of the human interferon beta counterpart. The sequence for this protein is disclosed at https://go.drugbank.com under accession number: DB09122.

Ofatumumab, as disclosed in U.S. Pat. No. 8,337,847, is a human monoclonal antibody for the CD20 protein. Ofatumumab binds specifically to both the small and large extracellular loops of the CD20 molecule. The CD20 molecule is expressed on normal B lymphocytes (pre-B- to mature B-lymphocyte) and on B-cell CLL. The Fab domain of ofatumumab binds to the CD20 molecule and the Fc domain mediates immune effector functions to result in B-cell lysis in vitro. Ofatumumab received FDA approval on Apr. 17, 2014, for use in combination with chlorambucil, for the treatment of previously untreated patients with CLL for whom fludarabine-based therapy is considered inappropriate. Ofatumumab was also approved by Health Canada on Aug. 13, 2012. The sequence for this protein is disclosed at https://go.drugbank.com under accession number: DB06650.

Obiltoxaximab, an affinity-enhanced monoclonal antibody (Mab), is used for prevention and treatment of infection and death caused by anthrax toxin. Obiltoxaximab is a chimeric IgG1 kappa monoclonal antibody (mAb) that binds the PA component of B. anthracis toxin. It has an approximate molecular weight of 148 kDa. The sequence for this protein is disclosed at https://go.drugbank.com under accession number: DB05336.

Nivolumab, as disclosed in patent US2013173223, is a fully human IgG4 monoclonal antibody that acts as an immunomodulator by blocking ligand activation of programmed cell death 1 (PD-1) receptor on T cells. It is indicated for use in patients with unresectable (cannot be surgically removed) or metastatic melanoma who no longer respond to other drugs. Nivolumab is administered as an intravenous infusion over 60 minutes every 2 weeks. The sequence for this protein is disclosed at https://go.drugbank.com under accession number: DB09035.

Necitumumab is an intravenously administered recombinant monoclonal IgG1 antibody used in the treatment of non-small cell lung cancer (NSCLC) as an EGFR antagonist. It functions by binding to epidermal growth factor receptor (EGFR) and prevents binding of its ligands, a process that is involved in cell proliferation, metastasis, angiogenesis, and malignant progression. Binding of necitumumab to EGFR induces receptor internalization and degradation, thereby preventing further activation of EGFR which is beneficial in NSCLC as many patients have increased protein expression of EGFR. Necitumumab is approved for use in combination with cisplatin and gemcitabine as a first-line treatment for metastatic squamous non-small cell lung cancer (NSCLC). The sequence for this protein is disclosed at https://go.drugbank.com under accession number: DB09559.

Mepolizumab, as disclosed in patent US2008134721, is a humanized IL-5 antagonist monoclonal antibody produced by recombinant DNA technology in Chinese hamster ovary cells. It has a molecular weight of approximately 149 kDa. It was approved by the FDA in November, 2015 for the treatment of asthma under the brand name Nucala (marketed by GlaxoSmithKline). Mepolizumab has been investigated in the treatment of severe nasal polyposis, among numerous other conditions. The sequence for this protein is disclosed at https://go.drugbank.com under accession number: DB06612.

Ixekizumab is a humanized immunoglobulin G subclass 4 (IgG4) monoclonal antibody (mAb) with neutralizing activity against IL-17A. Ixekizumab is produced by recombinant DNA technology in a recombinant mammalian cell line and purified using standard technology for bioproces sing. Ixekizumab is comprised of two identical light chain polypeptides of 219 amino acids each and two identical heavy chain polypeptides of 445 amino acids each, and has a molecular weight of 146, 158 Daltons for the protein backbone of the molecule. The sequence for this protein is disclosed at https://go.drugbank.com under accession number: DB11569.

Brodalumab has been used in trials studying the treatment of Asthma, Psoriasis, Crohn's Disease, Psoriatic Arthritis, and Rheumatoid Arthritis. Brodalumab was FDA approved in February, 2017 as Siliq for the treatment of moderate-to-severe plaque psoriasis. The sequence for this protein is disclosed at https://go.drugbank.com under accession number: DB11776.

C1 Esterase Inhibitor (Recombinant), as disclosed in patent NA, C1 Esterase Inhibitor (Recombinant) is a recombinant analogue of endogenous complement component-1 esterase inhibitor (rhC1INH), purified from the milk of transgenic rabbits. The primary function of endogenous C1INH is to regulate the activation of the complement and contact system pathways. It does this through inhibition of several target proteases within these pathways including activated C1 s, kallikrein, factor XIIa and factor XIa. C1 esterase inhibitor has also been shown to inhibit the action of thrombin within the coagulation pathway, and tPA and plasmin within the fibrinolytic pathway. Deficiency of C1-inhibitor permits plasma kallikrein activation, which leads to the production of the vasoactive peptide bradykinin. Additionally, C4 and C2 cleavage goes unchecked, resulting in auto-activation of the complement system. Down-stream effects of the lack of enzyme inhibition by C1 esterase inhibitor results in swelling due to leakage of fluid from blood vessels into connective tissue and consequently the presentation of hereditary angioedema (HAE). Marketed as the product Ruconest (FDA), this drug is indicated for the treatment of acute attacks of hereditary angioedema (HAE) due to C1 esterase inhibitor deficiency in adults. Intravenous replacement of C1 esterase inhibitor results in reversal of acute symptoms of HAE. The sequence for this protein is disclosed at https://go.drugbank.com under accession number: DB09228.

Dinutuximab, as disclosed in patent US20140170155, is an IgG1 monoclonal human/mouse chimeric antibody against GD2, a disialoganglioside expressed on tumors of neuroectodermal origin, including human neuroblastoma and melanoma, with highly restricted expression on normal tissues. It is composed of the variable heavy- and light-chain regions of the murine anti-GD2 mAb 14.18 and the constant regions of human IgG1 heavy-chain and kappa light-chain. By binding to GD2, dinutiximab induces antibody-dependent cell-mediated cytotoxicity and complement-dependent cytotoxicity of tumor cells thereby leading to apoptosis and inhibiting proliferation of the tumour. It is indicated, in combination with granulocyte-macrophage colony-stimulating factor (GM-CSF), interleukin-2 (IL-2), and 13-cis-retinoic acid (RA), for the treatment of pediatric patients with high-risk neuroblastoma who achieve at least a partial response to prior first-line multiagent, multimodality therapy. Despite a high clinical response seen after first-line treatment, the complete eradication of neuroblastoma is rarely achieved and the majority of patients with advanced disease suffer a relapse. Current strategies for treatment include immunotherapy with drugs such as dinutuximab to target surviving neuroblastoma cells and to prevent relapse. The sequence for this protein is disclosed at https://go.drugbank.com under accession number: DB09077.

Efmoroctocog alfa is a long-acting, fully-recombinant factor VIII Fc fusion protein. The sequence for this protein is disclosed at https://go.drugbank.com under accession number: DB11607.

Ibritumomab tiuxetan, as disclosed in patent CA2149329, is Indium or yttrium conjugated murine IgG1 kappa monoclonal antibody directed against the CD20 antigen, which is found on the surface of normal and malignant B lymphocytes. Ibritumomab is produced in Chinese hamster ovary cells and is composed of two murine gamma 1 heavy chains of 445 amino acids each and two kappa light chains of 213 amino acids each. The sequence for this protein is disclosed at https://go.drugbank.com under accession number: DB00078.

Lenograstim is a recombinant granulocyte colony-stimulating factor which functions as an immunostimulator. The sequence for this protein is disclosed at https://go.drugbank.com under accession number: DB13144.

Pegloticase is a recombinant procine-like uricase drug indicated for the treatment of severe, treatment-refractory, chronic gout. Similarly to rasburicase, pegloticase metabolises the conversion of uric acid to allantoin. This reduces the risk of precipitate formation and development of gout, since allantoin is five to ten times more soluble than uric acid. In contrast to rasburicase, pegloticase is pegylated to increase its elimination half-life from about eight hours to ten or twelve days, and to decrease the immunogenicity of the foreign uricase protein. This modification allows for an application just once every two to four weeks, making this drug suitable for long-term treatment. The sequence for this protein is disclosed at https://go.drugbank.com under accession number: DB09208.

Protein S (also known as S-Protein) is a vitamin K-dependent plasma glycoprotein synthesized in the liver. In the circulation, Protein S exists in two forms: a free form and a complex form bound to complement protein C4b-binding protein (C4BP). In humans, protein S is encoded by the PROS1 gene. The sequence for this protein is disclosed at https://go.drugbank.com under accession number: DB13149.

Somatropin recombinant, as disclosed in patents CA1326439, CA2252535, U.S. Pat. Nos. 5,288,703, 5,849,700, 5,849,704, 5,898,030, 6,004,297, 6,152,897, 6,235,004, 6,899,699, is a recombinant human growth hormone (somatotropin) 191 residues, MW 22.1 kD, synthesized in E. coli, The sequence for this protein is disclosed at https://go.drugbank.com under accession number: DB00052.

Susoctocog alfa is a recombinant, B-domain deleted, porcine sequence antihaemophilic factor VIII (FVIII) product that has recently been approved for the treatment of bleeding episodes in adults with acquired haemophilia A (AHA)., The sequence for this protein is disclosed at https://go.drugbank.com under accession number: DB11606.

Thrombomodulin Alfa is a recombinant and soluble thrombomodulin (ART-123). It is a human protein with both thrombin inhibiting and protein C stimulating activities, for the potential treatment of thromboembolism and blood clotting disorders, such as disseminated intravascular thromboembolism. The sequence for this protein is disclosed at https://go.drugbank.com under accession number: DB05777.

Applicants have isolated and identified novel sequence elements that can improve expression of recombinant proteins from at least two to ten-fold in stable cell lines when inserted in an expression vector. We refer to these novel sequence elements as GADS for genomic amplification drop sites.

GADS according to the instant disclosure have been found in a variety of mammalian genomes and cell lines. Some are found in many species and some are found in only human cells.

The novel insertion and amplification sites according to the disclosure comprise sequences that are found at sites that are adjacent to each other in wild type human cells, and some such sequences are found in the intergenic spacer regions of rDNA gene sequences. The useful GADS sequences were discovered in a research program to uncover the best possible insertion and amplification sites for recombinant protein.

Methods according to the instant disclosure involve the novel use of sequences hypothesized to be used by mammalian cells to further chromosomal evolution. Sequences according to the instant disclosure are responsible for the amplification processes that occur in these vast rDNA regions in the continuously ongoing chromosomal evolution.

An extensive research program was conducted to uncover the exact DNA sequences that are responsible for the amplification process described above and this research surprising lead to the development of the instant recombinant protein expression disclosure. During the examination of the vast amount of possible DNA it was found that no rDNA gene sequences are involved in the process and so the search began for sequences in the non-coding intergenic spacers between the genes and upstream and downstream from the rDNA genes in the non-coding chromosomal regions. This massive search required the examination of hundreds of kilobases of DNA sequences.

The extensive research described above uncovered two human GADS sequences: the CDC27 pseudogene sequence and a 2993 base pair (bp) sequence. Further examination and utilization of the 2993 bp sequence determined that a smaller portion of the sequence (904 bp) works well in certain embodiments of the methods for targeting and amplification in the HEK293 cell line according to the present disclosure. Embodiments of the disclosure utilizing this smaller sequence are advantageous when the exogenous gene to be expressed is large, or when several genes are to be expressed at the same time in the same cell line. In certain embodiments up to 4 genes can be expressed from one plasmid of the disclosure. In other embodiments multiple expression vectors may be used to transform the cells, increasing the number of polypeptides that can be produced simultaneously.

The present disclosure relates to the identification of recombinant protein integration sites in a variety of host genomes, and the construction of homologous recombination vectors for achieving high, stable recombinant gene expression in mammalian cells.

A number of GADS sequences have been identified and characterized. These sequences are disclosed as SEQ ID NO:1, SEQ ID NO:2 and SEQ ID NO:3 herein and also referred to as GADS1, GADS2 and GADS3 respectively.

### GADS1: Human CDC27 Pseudogene

Certain embodiments of the instant disclosure include sequences known as the Homo sapiens cell division cycle 27 pseudogene (CDC27 pseudogene). This pseudogene has no previously known function. There are many versions of CDC27 pseudogenes which can be used in embodiments of the instant disclosure. CDC27 pseudogenes are found on at least 10 chromosomes in wild type human cells including chromosomes 2, 7, 13, 14, 15, 16, 20, 21, 22, and Y. CDC27 pseudogene 11 is found on chromosomes 7, 15, 16, 20, 21, 22, and Y in wild type human cells. However, in HEK293 cells CDC27 pseudogene 11 (hereinafter GADS1) sequences are found on 11 to 13 chromosomes by FISH analysis. The more widespread distribution in HEK293 is hypothesized to be the result of the altered genome found in this transformed cell line. The increased distribution of the GADS1 sequence in HEK293 cells is useful in certain embodiments of the disclosure because there are more target sites in these cultured cells. The same is true of CHO and mouse cell lines.

A preferred sequence according to certain embodiments of the instant disclosure includes the 1978 base pair CDC27 pseudogene 11 sequence (GADS1). The CDC27 pseudogene sequences according to the disclosure are useful in embodiments of the instant disclosure where protein production in cell lines originating from a wide variety of different species are desired because the CDC27 pseudogene sequence is highly conserved in a variety of mammalian species. Nucleotide sequence identity to Cricetulus griseus (Chinese Hamster) CDC27 pseudogene DNA sequence is 85.87% compared to the whole human sequence with only 1% gaps were detected in a Blast search. Nucleotide sequence identity to Mus musculus (mouse) CDC27 pseudogene DNA sequence is 84.65% compared to the whole human sequence with only 1% gaps were detected in a Blast search. The CDC27 pseudogene is even more highly conserved across different primate species.

GADS1 sequences according to the instant disclosure have been shown by FISH analysis to be present on at least 11 chromosomes in HEK293 cells, on the acrocentric arms in several hundred to thousands of copies. Thus, embodiments of the disclosure including constructs and methods that use these sequences are preferred for very high amplification and production of exogenous proteins.

GADS1 sequences useful according to the present disclosure have been identified on chromosomes 2, 22 and Y. (See RefSeq, accessed May 2014: https://www.alliancegenome.org/gene/HGNC:1728). In addition, GADS1 sequences are found in Nucleolus organiser regions (NORs) which are chromosomal regions crucial for the formation of the nucleolus. In humans, the NORs are located on the short arms of the acrocentric chromosomes 13, 14, 15, 21 and 22, the genes RNR1, RNR2, RNR3, RNR4, and RNR5 respectively. Finally, wild type human cells carry GADS1 sequences on Chromosomes 7, 15, 16, 20, 21, 22, Y. These regions carry tens of copies of this sequence, each. So, the wild type genome carries hundreds of copies of this sequence.

GADS1 sequences were successfully used in methods according to the present disclosure utilizing CHO-K1 cells where insertion and amplification of exogenous DNA was demonstrated. Targeting and amplification was demonstrated in three different regions on large metacentric hamster chromosomes: at the very end of metacentric chromosomes; in the middle of one chromosomal arm of metacentric chromosomes; and in the middle (close to the centromere) of metacentric chromosomes.

In other embodiments mouse GADS1 homologues may be used for exogenous gene expression in mouse cell lines.

SEQ ID NO:1 according to the present disclosure comprises the 5′-3′ DNA sequence disclosed in the Sequence Listing provided herewith, originating from Homo sapiens and having a length of 1978 bp (“SEQ ID NO:1” and “GADS1” are used interchangeably herein).

In preferred embodiments of the instant disclosure GADS1 (SEQ ID NO:1) sequences are found in euchromatin regions, suitable for expression and are not silenced. This makes these sites ideal for recombinant protein production. There are many sites in the target genome where the gene of interest can be integrated with the help of CDC27 pseudogenes such as the preferred sequence of GADS1.

As discussed above and while not wanting to be bound by theory, the natural CDC27 pseudogenes are hypothesized to have an evolutionary function, because they are so extremely conserved. This CDC27 pseudogene sequence is a natural amplificator. In certain embodiments the application of antibiotic selection pressure to this sequence causes the GADS to be highly amplified together with the gene of interest.

The chromatin around the CDC27 pseudogenes is always open for gene expression making the sites extremely useful in certain embodiments of the disclosure for recombinant gene expression. The inventors hypothesize that these and the other identified GADS sequences are involved in chromosomal evolution and have evolved to essentially be “untouchable” zones—remaining intact throughout evolutionary history. In addition, the natural amplification of these GADS, is accomplished without silencing, setting the systems of the present disclosure apart from all of the other amplification and expression systems previously developed.

Further, a significant advantage of embodiments of the present disclosure over the previous known DHFR and GS based amplification processes that are used in the industry presently, is that the amplified chromosome arm created in embodiments of the present disclosure are stable and that is one reason why recombinant proteins are expressing stably at high levels in embodiments of the present disclosure.

In addition to GADS1, the present disclosure encompasses fragments of SEQ ID NO:1 that also exhibit GADS activity.

Expression vectors comprising the isolated 1978 bp sequence (SEQ ID NO:1) and shorter fragments thereof are useful to transform CHO cells and result in high levels of stable protein expression. The present novel GADS1 is useful to improve expression of a recombinant protein driven by a promoter/enhancer region to which it is linked.

Expression vectors comprising the isolated 1978 bp sequence (SEQ ID NO:1) and shorter fragments thereof are useful to transform HEK293 cells and result in high levels of stable protein expression. The present novel GADS1 is useful to improve expression of a recombinant protein driven by a promoter/enhancer region to which it is linked.

Moreover, additional fragments of SEQ ID NO:1 exhibiting GADS activity can be identified, as well as similar GADS motifs from other types of cells or from other integration sites in transformed cells. In addition, it is known in the art that subsequent processing of fragments of DNA prepared by restriction enzyme digestion can result in the removal of additional nucleotides from the ends of the fragments.

A fragment (211 bp—SEQ ID NO:4) of GADS1 (SEQ ID NO:1) is also a part of the 904 bp sequence of GADS3 (SEQ ID NO:3) and also the part of the 2993 bp sequence GADS2 (SEQ ID NO:2). One skilled in the art can thus devise various fragments of the sequences disclosed herein for use in additional embodiments of the present disclosure.

Other combinations of fragments of SEQ ID NO:1 can also be developed, for example, sequences that include multiple copies of all or a part of SEQ ID NO:1. Such combinations can be contiguously linked or arranged to provide optimal spacing of the fragments. Additionally, within the scope of the present disclosure are expression vectors comprising the sequence of SEQ ID NO:1 arranged with insertion sequences therein (e.g., insertion of a gene encoding a desired protein at a certain selected site in SEQ ID NO:1).

### GADS2: 2993 bp Long Sequence

In additional embodiments of the instant disclosure a 2993 bp sequence with no previously known function, hereinafter called “GADS2” is provided. GADS2 sequences are found in wild type human cells on chromosomes 7, 15, 16, 20, 21, 22 and Y. These regions each carry tens of copies of this sequence. Thus wild-type human genomes carry hundreds of copies from this sequence. A small part of this sequence overlaps with the sequence of GADS1.

There is no known equivalent sequence in non-human species to GADS2 but this result might be merely because the non-coding portions of these genomes have been less well characterized.

In certain embodiments the GADS2 sequence was used to make targeting and amplification of plasmids in mouse and hamster cell lines.

GADS2: SEQ ID NO:2 according to the present disclosure comprises the 5′-3′ DNA sequence disclosed in the Sequence Listing provided herewith, originating from Homo sapiens and having a length of 2993 bp (“SEQ ID NO:2” and “GADS2” are used interchangeably herein).

### GADS3: 904 bp Long Sequence (a Smaller Fragment of the 2993 bp Sequence)

A smaller part of the GADS2 sequence is especially useful in developing human cell lines expressing recombinant proteins. The smaller sequence comprises 904 base pairs of the GADS2 sequence and is referred to as GADS3 or SEQ ID NO:3. Wild type human cells carry these GADS3 sequences on chromosomes 7, 15, 16, 20, 21, 22, and Y. These regions carry tens of copies of this GADS3 sequence, each. The wild-type human genome carries hundreds of copies of GADS3 sequences.

Cultured human cells (including but not limited to HEK293 cells) carry hundreds to thousands of copies from this GADS3 sequence based on FISH experiments. As discussed previously, cultured human cells have altered genomes, chromosome numbers and chromosome rearrangements and amplifications are very frequent events. Thus, GADS3 sequences are ideal for certain preferred embodiments of the methods and constructs of the instant disclosure.

GADS3 has a number of desirable characteristics for use in preferred methods and constructs of the instant disclosure. 211 bp of the GADS3 sequence overlaps with GADS1 at the 5′ end.

No GADS3 nucleotide sequence identity was found to Cricetulus griseus Blast sequences.

No GADS3 nucleotide sequence identity was found to Mus musculus Blast sequences. Thus, in certain embodiments of the disclosure, methods and constructs incorporating GADS3 sequences are useful for production and amplification in human cell lines.

One significant advantage of embodiments of the instant disclosure comprising the GADS3 sequence is that GADS3 is small which is advantageous when you have large exogenous genes to express, or several genes to be expressed at the same time. Certain embodiments comprising expression vectors including GADS3 can include up to 4 exogenous genes to be expressed on a single plasmid. As disclosed in the examples, GADS3 vectors are useful for simultaneous expression of both the heavy and light chains of antibodies.

GADS3: SEQ ID NO:3 according to the present disclosure comprises the 5′-3′ DNA sequence disclosed in the Sequence Listing provided herewith, originating from Homo sapiens and having a length of 904 bp (“SEQ ID NO:3” and “GADS3” are used interchangeably herein).

### GADS4: SEQ ID NO:4—a 211 bp Fragment of GADS2 and GADS3

There is a 211 bp overlap between CDC27 pseudogene 11 (GADS1, SEQ ID NO:1), the 904 bp sequence (GADS3, SEQ ID NO:3) and the 2993 bp sequence (GADS2, SEQ ID NO:2). This sequence, hereinafter “GADS4” or “SEQ ID NO:4”, can be used in certain embodiments of the disclosure.

GADS4: SEQ ID NO:4 according to the present disclosure comprises the 5′-3′ DNA sequence disclosed in the Sequence Listing provided herewith, originating from Homo sapiens and having a length of 211 bp (“SEQ ID NO:4” and “GADS4” are used interchangeably herein).

### GADS5: SEQ ID NO:5—a 293 bp Fragment of GADS2

A fragment of the 2993 bp human sequence (GADS2, SEQ ID NO:2) has a 238 bp identity with 2% gaps with mouse genomic sequence. This sequence does not overlap with the 904 bp sequence (GADS3, SEQ ID NO:3) or with the CDC27 pseudogene 11 sequence (GADS1, SEQ ID NO:1).

GADS5: SEQ ID NO:5 according to the present disclosure comprises the 5′-3′ DNA sequence disclosed in the Sequence Listing provided herewith, originating from Homo sapiens and having a length of 293 bp (“SEQ ID NO:5” and “GADS5” are used interchangeably herein).

One skilled in the art will recognize that changes can be made in the nucleotide sequences set forth in SEQ ID NO:1, SEQ ID NO:2 and SEQ ID NO:3 by site directed or random mutagenesis techniques that are known in the art. The resulting GADS variants can then be tested for GADS homologous recombination activity as described herein. DNAs that are at least about 80%, more preferably about 85%, and more preferably about 90% identical in nucleotide sequence to SEQ ID NO: 1, 2, or 3 or fragments thereof, having GADS homologous recombination activity are isolatable by experimentation and hypothesized to have GADS recombination. Accordingly, homologues of the disclosed GADS sequences and variants thereof are also encompassed by the present disclosure.

The following examples are illustrative of embodiments of the present disclosure and do not limit the scope of the disclosure in any way. All references cited herein, whether supra or infra, are hereby incorporated by reference in their entirety.

### EXAMPLES

### Example 1: GADS3 CHO-K1 Cell Line Production of Trastuzumab

The optimal DNA sequence for expression of trastuzumab was determined and the DNA was synthesized in a pUC plasmid and transferred into an appropriate insertion vector according to the disclosure for insertion in CHO-K1 cells. A typical plasmid is shown in FIG. 1.

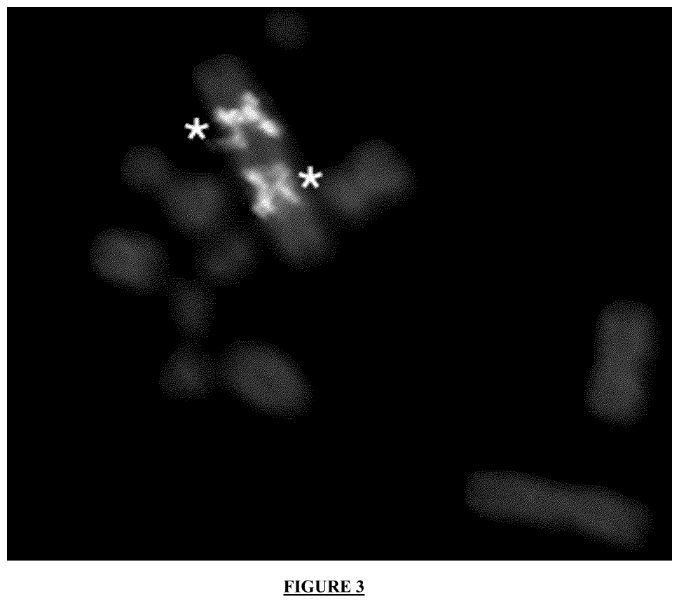

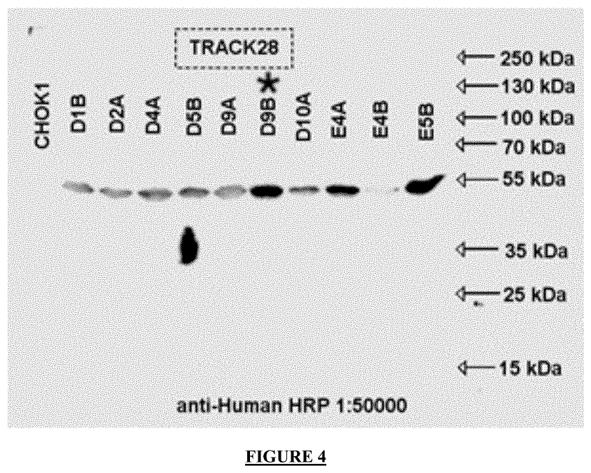

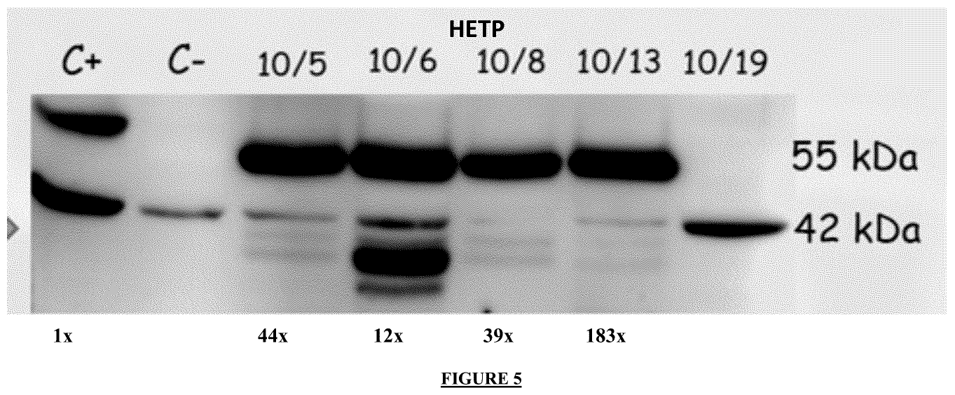

This plasmid was used to establish stable CHO-K1 cell lines with amplified “trastuzumab” as follows:
a. CHO-K1 cells were seeded into one well of a 24-well plate (approximately 200000 cells). The next day cells were transfected with 1 μg trastuzumab containing plasmid. For this cell line we use the Turbofect reagent (see https://www.thermofisher.com/order/catalog/product/R0532 #/R0532, accessed May 31, 2021).
b. The insertion plasmid was diluted with 100 μl serum-free DMEM medium.
c. The Turbofect reagent was mixed thoroughly by vortexing.
d. 2 μl Turbofect reagent was added to the insertion plasmid solution.
e. This reaction was mixed by pipetting and incubated at room temperature for 20 minutes.
f. The transfection mix was added evenly and drop by drop to the 24 cells in the wells with each well containing serum containing 1 ml DMEM medium.
g. After 24 hours, the cells were collected using TrypLE Select reagent and then distributed into the wells of 2×96-well plates. About 40 ml of DMEM was used in order to achieve volume of about 200 μl×96×2 ml altogether.
h. After 24 hours antibiotic selection is begun by the addition of 10 μg/ml puromycin.
i. After 3 days the selection medium is exchanged for fresh medium.
j. After an additional 3 days the selection medium is again exchanged.
k. After an additional 3 days the growing cell clones are collected from the wells of the 96-well plate by TrypLE Select reagent, and individually transferred into one well of a 24-well plate.
l. 24 hours later antibiotic selection with 50 ug/ml Puromycin is started.
m. 3 days later the selection medium is exchanged for medium containing 100 μg/ml Puromycin containing medium.
n. The growing cell lines were examined by lysing the cells and purifying the total protein. Western blot analysis (see FIG. 4) was used to determine the production of trastuzumab.
o. The highest producing cell lines are checked with FISH experiments for the presence of the insertion plasmid. See, for example, FIG. 3.
p. The best clones are further selected with increased antibiotic selection. The cells that are growing at 100 μg/ml puromycin are split into 3 wells of a 24-well plate and after 24 hours the cells are subjected to 150 μg/ml, 175 μg/ml and 200 μg/ml puromycin.
q. Surviving clones are examined with western blotting for protein production and with FISH for the presence of amplification.
r. The best producing clones are grown in serum-free medium.
s. Trastuzumab is purified from the medium.
t. The concentration of trastuzumab is determined by spectrophotometer, HPLC or similar procedures.
u. Highest yielding cell lines will be selected and trastuzumab can be purified.

### Example 2: GADS3 HEK293 Cell Line Production of Trastuzumab

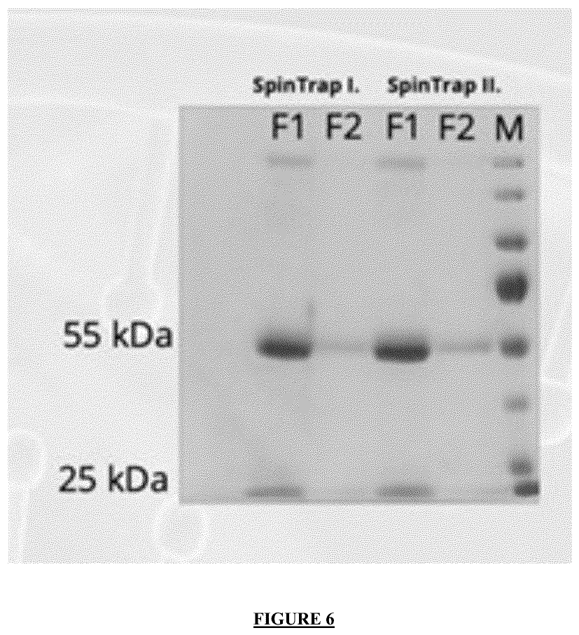

Plasmids containing the GADS3 sequence (SEQ ID NO:3) were used to transfect HEK293 cell line. The insertion vector is shown in FIG. 2. The production of trastuzumab was confirmed by Western blotting as shown in FIG. 5 using the 42 kDa actin protein expression for quantitation.

Trastuzumab is purified using protein G columns. The Protein G HP SpinTrap column (Merck) was used for small scale purification. FIG. 6 shows the purified trastuzumab both heavy and light chains on a Coomassie blue stained acrylamide gel. This staining shows the actual protein which was purified on the protein G column (HETP10/13 Herceptin; 0.9-3 g/L (Bradford); 3.5×106 cell/column: 111 pg/cell). (for SpinTrap see https://www.sigmaaldrich.com/catalog/product/si gma/ge28903134?lang=hu&region=HU&gclid=CjwKCAjwtdeFBhBAEiwAKOIy5914n5X1r4X4s11mGD_Z46KOJtAlk-r1omujFYESnStEUWOXp-qqThoCj-4QAvD_BwE, accessed May 31, 2021).

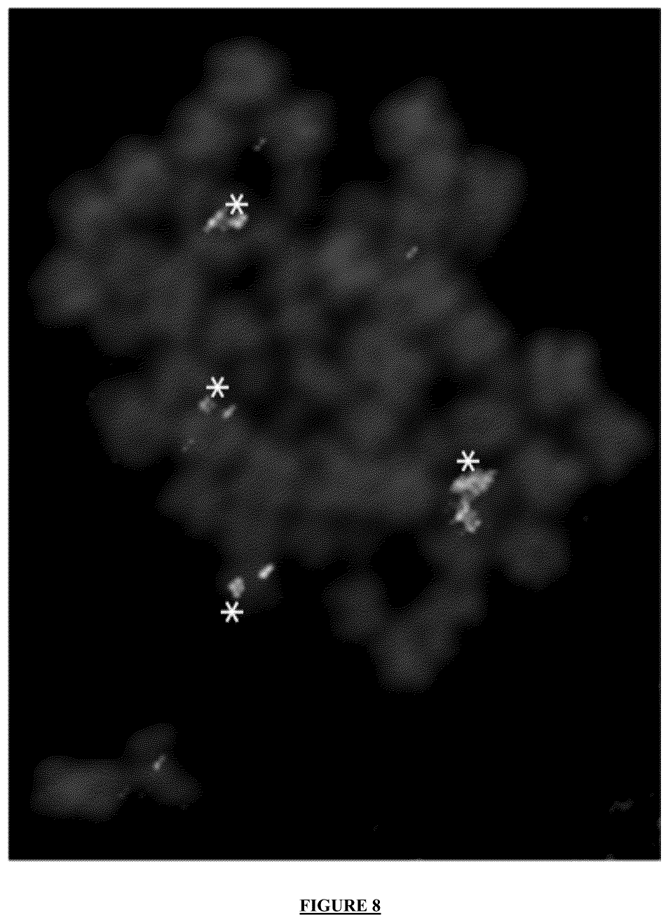

Based on the Bradford protein concentration measurement, we found that trastuzumab production was at least 111 pg/cell. 5 [239] The highest producing cell line (HETP10/13) was subjected to subcloning (see FIG. 7) and the highest producing clone (#11) was subjected to FISH analysis (see FIG. 8).

### Example 3: GADS2 Cell Line Production in Mouse Cell Lines

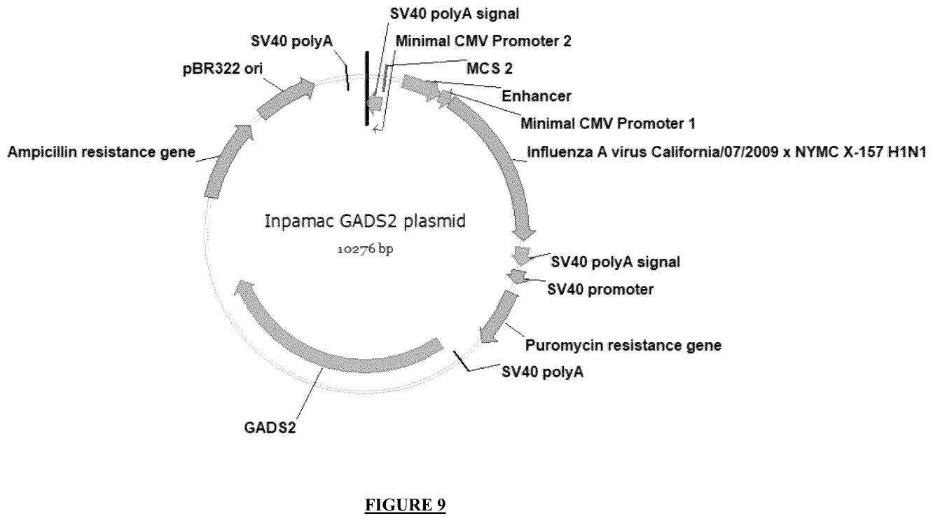

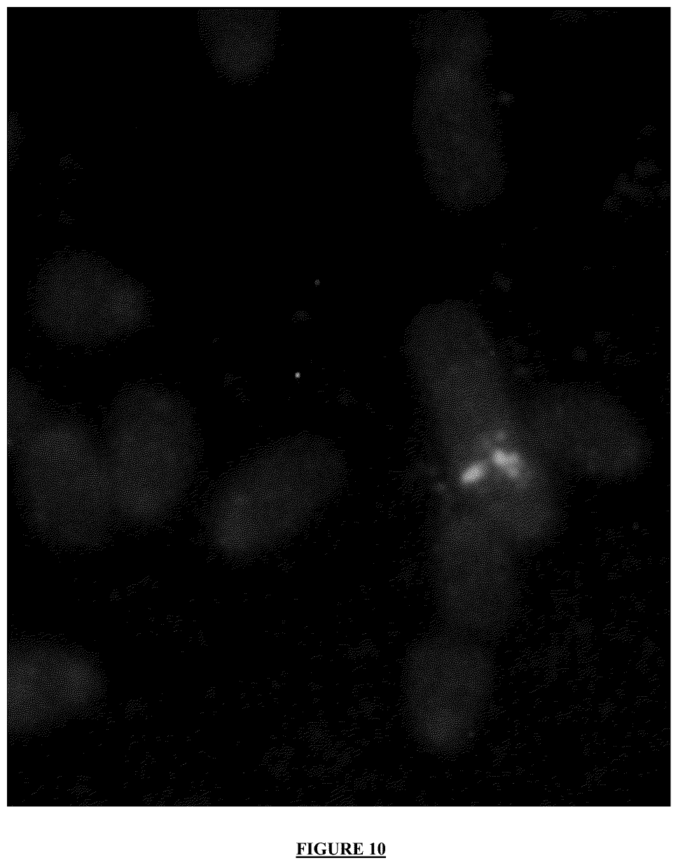

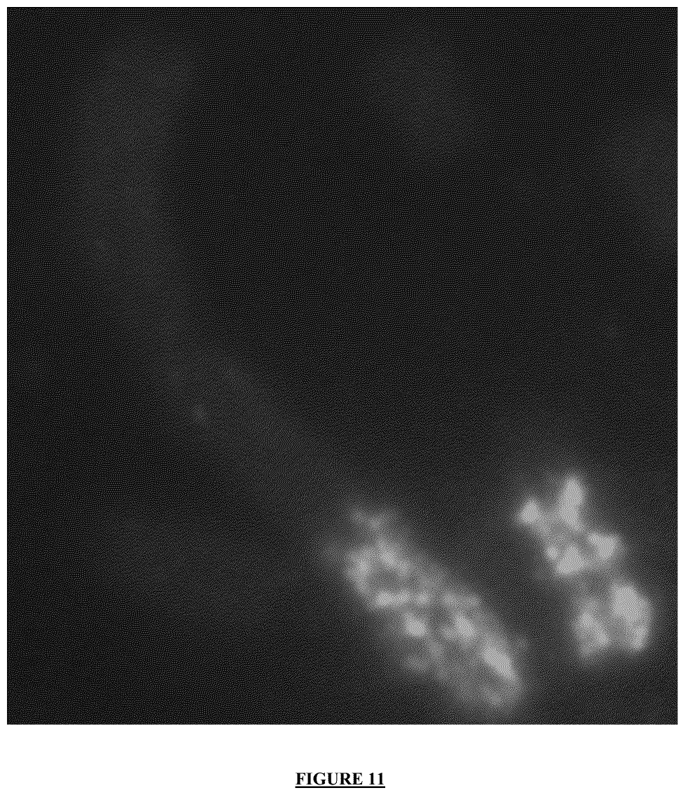

Mouse cell lines: Mouse embryonic fibroblast (MEF) cells from 3.5 days old individual mouse embryos were isolated and used to establish stable cell lines. The cells were immortalized with well documented methods: basically, cells were passaged every 3 days until immortalized. These cells show several markers of mesenchymal stem cells by FACS experiments. The first step, shown here, demonstrates a vector according to the disclosure that carried the 2993 bp sequence (GADS2: SEQ ID NO:2) into which was inserted a useful gene (Influenza A virus Hemagglutinin MYMC_X-181 California strain) as shown in FIG. 9. This embodiment of the disclosure was transfected into the immortalized MEFs, producing 34 stable cell lines with small-scale amplification that resulted in 30-50 copies of the exogenous sequence, as shown in FIG. 10. Surprisingly, targeting exclusively happened into a large acrocentric chromosome in the upper part of the long chromosomal arm in all 34 clones.

### Example 4: GADS2 Cell Line Production in CHO Cell Lines

A plasmid according to the disclosure (shown in FIG. 9) was used to transfect CHO-DG44 cells producing 79 clones.

### Example 5: GADS1 Cell Line Production in Mouse Cell Lines

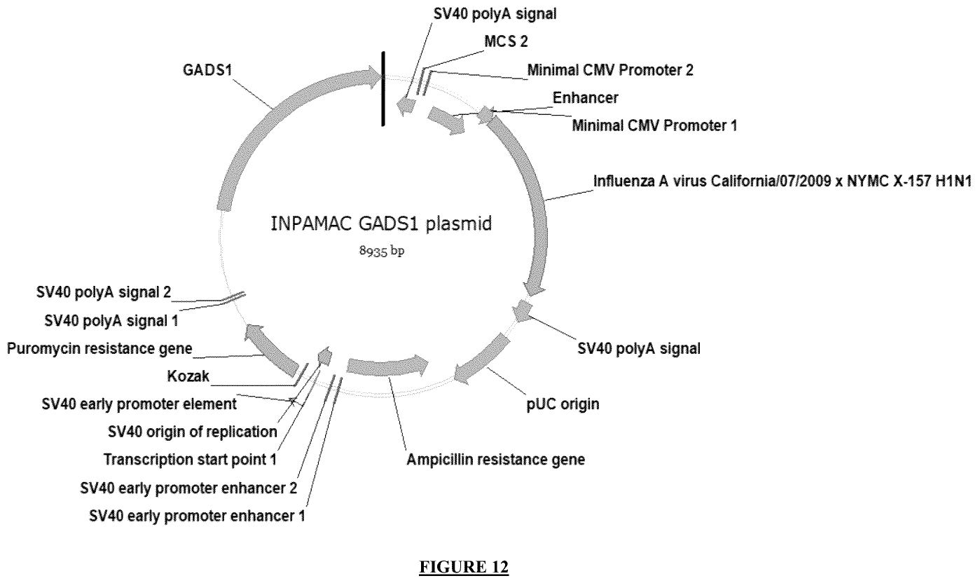

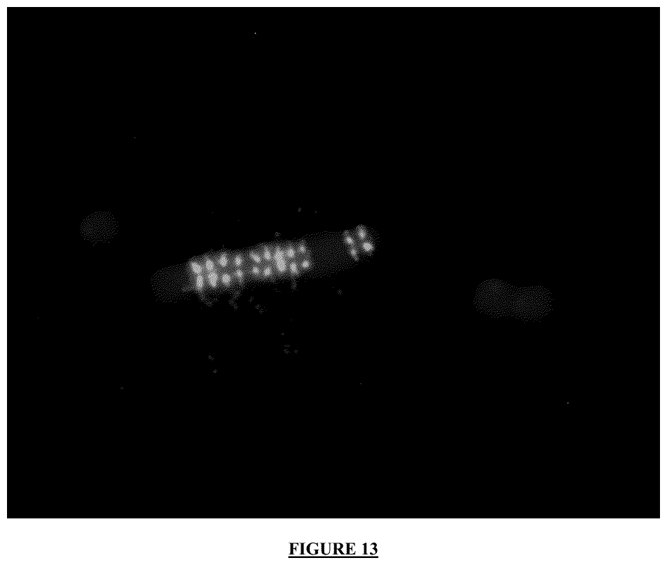

LMTK-mouse cells were transfected with a plasmid construct according to the disclosure carrying GADS1 sequence (CDC27 pseudogene: SEQ ID NO:1) and the Influenza A virus Hemagglutinin MYMC_X-181 California strain sequence was inserted as a useful exogenous gene (FIG. 12). We produced 32 cell lines overexpressing the transgene. FIG. 13 shows FISH experiment carried out with the plasmid on FIG. 12. Plasmid was labeled with a green fluorescent dye. Green staining shows the presence of the transgenes on a mouse chromosome in bands (several hundred copies). Mouse chromosomes were counterstained with DAPI (blue fluorescent dye).

### Example 6: GADS1 Cell Line Production in Mouse Cell Lines

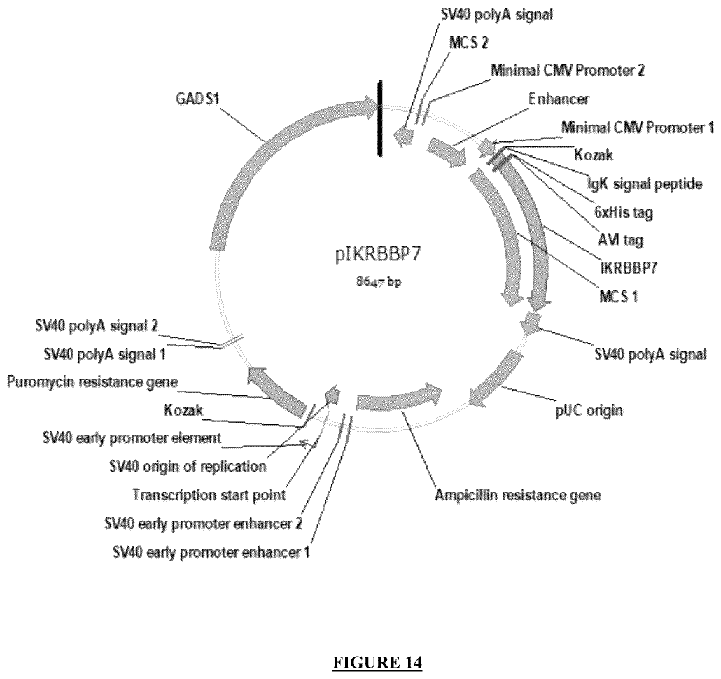

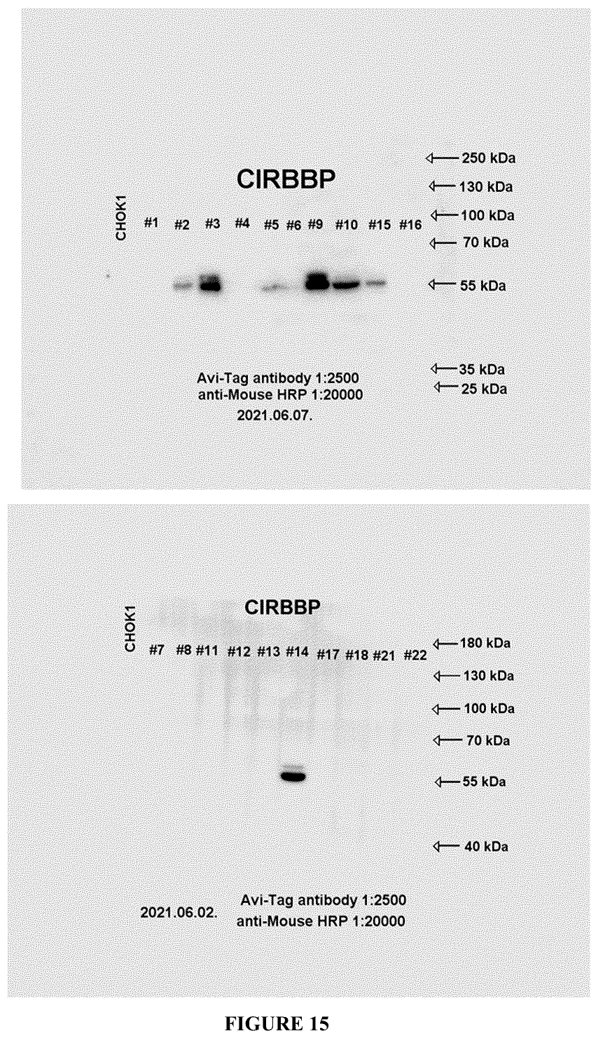

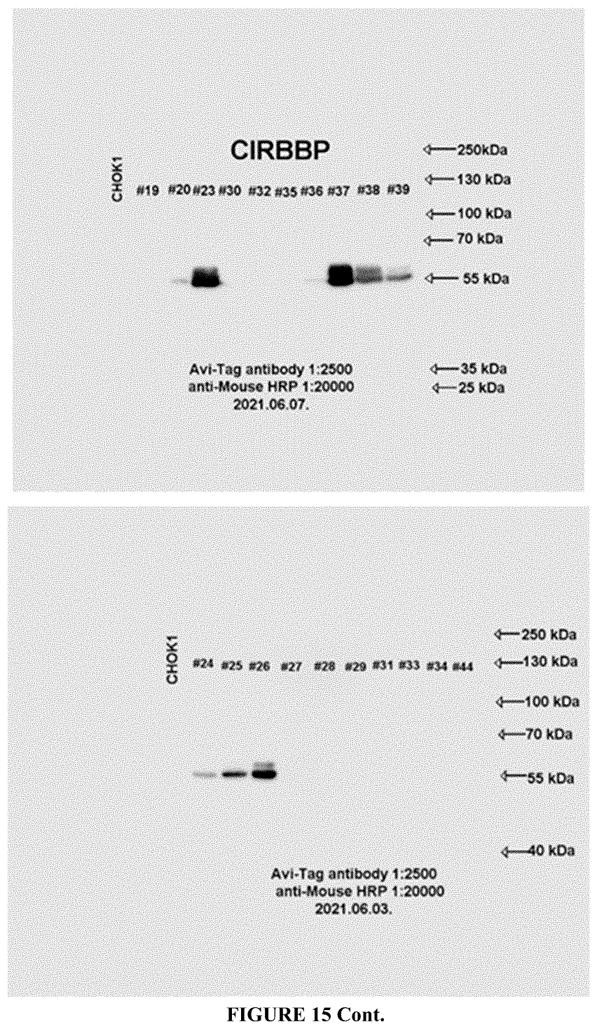

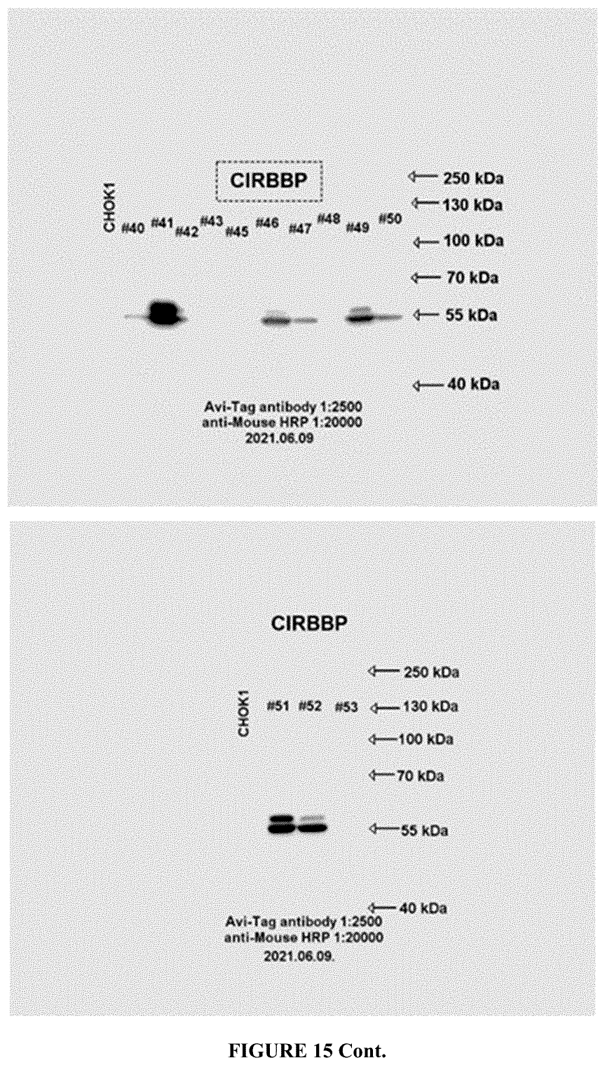

CHO-K1 cell lines were made with a plasmid according to the disclosure carrying the GADS1 sequence (CDC27 pseudogene: SEQ ID NO:1) and expressing RBBP7 protein (pIKRBBP7, see FIG. 14). Two types of cell lines were constructed in this embodiment. One type of cell line expresses and secretes the RBBP7 protein into the culture medium (CIRBBP cell lines). In this embodiment, the RBBP7 protein is expressed with a hamster IgK secretion signal on the N-terminal. After the secretion signal, there are two tags for labeling and purification (an AVI tag and a 6×His tag). 53 cell lines were produced with this construct and 24 of them stably produce the RBBP7 protein (see FIG. 15). This was shown by Western blotting experiments.

### Example 7: GADS1 Cell Line Production in Mouse Cell Lines

Additional RBBP7 protein producing cell lines were produced without the hamster IgK secretion signal to cause the protein to remain inside the cells rather than be excreted. Proteins produced in this embodiment are purified from cell lysates.

While the present disclosure describes various embodiments for illustrative purposes, such description is not intended to be limited to such embodiments. On the contrary, the applicant's teachings described and illustrated herein encompass various alternatives, modifications, and equivalents, without departing from the embodiments, the general scope of which is defined in the appended claims. Except to the extent necessary or inherent in the methods or processes themselves, no particular order to steps or stages of methods or processes described in this disclosure is intended or implied. In many cases the order of method or process steps may be varied without changing the purpose, effect, or import of the methods described.

Information as herein shown and described in detail is fully capable of attaining the above-described object of the present disclosure, the presently preferred embodiment of the present disclosure, and is, thus, representative of the subject matter which is broadly contemplated by the present disclosure. The scope of the present disclosure fully encompasses other embodiments which may become apparent to those skilled in the art, and is to be limited, accordingly, by nothing other than the appended claims, wherein any reference to an element being made in the singular is not intended to mean “one and only one” unless explicitly so stated, but rather “one or more.” All structural and functional equivalents to the elements of the above-described preferred embodiment and additional embodiments as regarded by those of ordinary skill in the art are intended to be encompassed by the present claims. Moreover, no requirement exists for a system or method to address each and every problem sought to be resolved or ameliorated by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, as may be apparent to those of ordinary skill in the art, are also encompassed by the disclosure.

What is claimed is:

1. An expression vector comprising a DNA fragment capable of targeted insertion into a region of open chromatin in mammalian cells on at least one chromosome at more than one site wherein the DNA fragment includes:
a. a selectable marker,
b. an amplification sequence comprising SEQ ID NO:1, SEQ ID NO:2, SEQ ID NO:3, SEQ ID NO:4, SEQ ID NO:5, or a functional fragment thereof, and
c. further comprising a nucleic acid sequence encoding all or a portion of a protein of interest.

2. The expression vector of claim 1, wherein the selectable marker provides resistance to puromycin.

3. The expression vector of either one of claim 1 or claim 2, further comprising a nucleic acid sequence encoding all or a portion of a protein of interest.

4. The expression vector of claim 3, wherein said protein of interest is a drug selected from the group consisting of adalimumab, atezolizumab, nivolumab, pembrolizumab, etanercept, trastuzumab, bevacizumab, rituximab, aflibercept, infliximab, ustekinumab, ranibizumab, cetuximab, dornase alfa, peginterferon alfa-2a, darbepoetin alfa, reteplase, epoetin alfa, pegfilgrastim, thyrotropin alfa, antihemophilic factor, anistreplase, tenecteplase, coagulation factor viia, omalizumab, imiglucerase, abciximab, abciximab, interferon beta-1a, follitropin beta, basiliximab, muromonab, ibritumomab, tositumomab, alemtuzumab, laronidase, efalizumab, choriogonadotropin alfa, coagulation factor ix, agalsidase beta, palivizumab, daclizumab, arcitumomab, eculizumab, panitumumab, idursulfase, alglucosidase alfa, galsulfase, abatacept, canakinumab, ipilimumab, tocilizumab, pertuzumab, rilonacept, denosumab, belatacept, belatacept, velaglucerase alfa, brentuximab vedotin, taliglucerase alfa, belimumab, raxibacumab, epoetin zeta, obinutuzumab, follitropin alpha, romiplostim, natalizumab, secukinumab, drotrecogin alfa, alefacept, urokinase, gemtuzumab ozogamicin, satumomab pendetide, alirocumab, ancestim, antithrombin alfa, antithrombin iii human, asfotase alfa, blinatumomab, c1 esterase inhibitor (human), coagulation factor xiii a-subunit (recombinant), conestat alfa, daratumumab, lotuzumab, evolocumab, hyaluronidase (human recombinant), idarucizumab, vedolizumab, turoctocog alfa, simoctocog alfa, siltuximab, sebelipase alfa, ramucirumab, peginterferon beta-1a, ofatumumab, obiltoxaximab, necitumumab, mepolizumab, ixekizumab, rodalumab, c1 esterase inhibitor (recombinant, canakinumab, dinutuximab, efmoroctocog alfa, ibritumomab tiuxetan, lenograstim, pegloticase, protein s human, somatropin recombinant, susoctocog alfa, or thrombomodulin alfa.

5. A mammalian host cell transformed with the expression vector of claim 4 wherein the cell contains at least 5 copies of the protein of interest.

6. The mammalian host cell transformed of claim 5, wherein the mammalian host cell is a HEK293 cell, a HT1080 cell, a Per.C6 cell, a pluripotent cell, an induced pluripotent cell, a totipotent cell, an adult stem cell, a cultured adult cell, an immortalized cell or a primary cell.

7. A Chinese hamster ovary (CHO) cell transformed with the expression vector of claim 4.

8. A mouse cell transformed with the expression vector of claim 4.

9. A human cell transformed with the expression vector of claim 4.

10. A method for obtaining a recombinant protein, comprising culturing a transformed host cell according to any one of claims 5 to 9, under conditions promoting expression of said recombinant protein, and recovering the recombinant protein.

11. A DNA vector capable of stable integration into a mammalian genome, said DNA vector comprising:
a. a DNA fragment capable of site-specific insertion a region of open chromatin in mammalian cells, wherein said DNA fragment is the sequence of SEQ ID NO:1, SEQ ID NO:2, SEQ ID NO:3, SEQ ID NO:4, SEQ ID NO:5, or a functional fragment thereof;
b. at least one transgene encoding at least one protein of interest selected from adalimumab, atezolizumab, nivolumab, pembrolizumab, etanercept, trastuzumab, bevacizumab, rituximab, aflibercept, infliximab, ustekinumab, ranibizumab, cetuximab, dornase alfa, peginterferon alfa-2a, darbepoetin alfa, reteplase, epoetin alfa, pegfilgrastim, thyrotropin alfa, antihemophilic factor, anistreplase, tenecteplase, coagulation factor viia, omalizumab, imiglucerase, abciximab, abciximab, interferon beta-1a, follitropin beta, basiliximab, muromonab, ibritumomab, tositumomab, alemtuzumab, laronidase, efalizumab, choriogonadotropin alfa, coagulation factor ix, agalsidase beta, palivizumab, daclizumab, arcitumomab, eculizumab, panitumumab, idursulfase, alglucosidase alfa, galsulfase, abatacept, canakinumab, ipilimumab, tocilizumab, pertuzumab, rilonacept, denosumab, belatacept, belatacept, velaglucerase alfa, brentuximab vedotin, taliglucerase alfa, belimumab, raxibacumab, epoetin zeta, obinutuzumab, follitropin alpha, romiplostim, natalizumab, secukinumab, drotrecogin alfa, alefacept, urokinase, gemtuzumab ozogamicin, satumomab pendetide, alirocumab, ancestim, antithrombin alfa, antithrombin iii human, asfotase alfa, blinatumomab, c1 esterase inhibitor (human), coagulation factor xiii a-subunit (recombinant), conestat alfa, daratumumab, lotuzumab, evolocumab, hyaluronidase (human recombinant), idarucizumab, vedolizumab, turoctocog alfa, simoctocog alfa, siltuximab, sebelipase alfa, ramucirumab, peginterferon beta-1a, ofatumumab, obiltoxaximab, necitumumab, mepolizumab, ixekizumab, brodalumab, c1 esterase inhibitor (recombinant, canakinumab, dinutuximab, efmoroctocog alfa, ibritumomab tiuxetan, lenograstim, pegloticase, protein s human, somatropin recombinant, susoctocog alfa, or thrombomodulin alfa to be expressed by a mammalian cell after the DNA vector integrates into the mammalian genome of the mammalian cell.
